SEQUENCE LISTING (45 sequences)

SEQ ID NO: 1 - protein, 275 aa, Bacillus subtilis
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 2 - protein, 244 aa, synthetic construct
SGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 3 - protein, 268 aa, synthetic construct
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 4 - protein, 237 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 5 - protein, 235 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 6 - protein, 235 aa, synthetic construct
LRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 7 - protein, 227 aa, synthetic construct
DAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 8 - protein, 225 aa, synthetic construct
AGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 9 - protein, 218 aa, synthetic construct
KMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 10 - protein, 221 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 11 - protein, 205 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 12 - protein, 231 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQN

SEQ ID NO: 13 - protein, 227 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQ

SEQ ID NO: 14 - protein, 197 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIR

SEQ ID NO: 15 - protein, 191 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTA

SEQ ID NO: 16 - protein, 178 aa, synthetic construct
KMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIR

SEQ ID NO: 17 - protein, 226 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEQASQAMLAQANQQPQNVLQLLR

SEQ ID NO: 18 - protein, 222 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDQASQAMLAQANQQPQNVLQLLR

SEQ ID NO: 19 - protein, 219 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEAQANQQPQNVLQLLR

SEQ ID NO: 20 - protein, 219 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRQASQAMLAQANQQPQNVLQLLR

SEQ ID NO: 21 - protein, 237 aa, synthetic construct
SGCRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRCVDMAKEMSEFTKNNILSQASQAMLAQANQQPQNVLQLLR

SEQ ID NO: 22 - protein, 237 aa, synthetic construct
SGLRINRAGDDAAGLAICEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAACSRIRDVDMAKEMSEFTKNNILSQASQAMLAQANQQPQNVLQLLR

SEQ ID NO: 23 - protein, 237 aa, synthetic construct
SGLRINRAGDDAAGLAISEKCRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAACSRIRDVDMAKEMSEFTKNNILSQASQAMLAQANQQPQNVLQLLR

SEQ ID NO: 24 - protein, 237 aa, synthetic construct
SGLRINRAGDDAAGLAISEKMRGQIRGCEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASCENLTAAESRIRDVDMAKEMSEFTKNNILSQASQAMLAQANQQPQNVLQLLR

SEQ ID NO: 25 - protein, 237 aa, synthetic construct
SGLRINRAGDDAAGLAISEKMRGQIRGLEMASKNSQDGISLIQTAEGALTETHAILQRMRELTVQAGNGTAAAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 26 - protein, 237 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIAANAGQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 27 - protein, 237 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAAQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 28 - protein, 237 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALAVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 29 - protein, 237 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVAMAKEMSEFTKNNILSQASQAMLAQANQQPQNVLQLLR

SEQ ID NO: 30 - protein, 237 aa, synthetic construct
SGLRINRAGDDAAGLAISEKMRGQIRGLEMASKNSQDGISLIQTAEGALTETHAILQRMRELTVQAGNGTAAAEDLGAIKDEMDALIEEIDGISNRTEFNGKKLLDGTNSTTFQIGANAAQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 31 - protein, 237 aa, synthetic construct
SGLRINRAGDDAAGLAISEKMRGQIRGLEMASKNSQDGISLIQTAEGALTETHAILQRMRELTVQAGNGTAAAEDLGAIKDEMDALIEEIDGISNRTEFNGKKLLDGTNSTTFQIGANAGQQLNVKIDSMSSTALGVNALAVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 32 - protein, 237 aa, synthetic construct
SGLRINRAGDDAAGLAISEKMRGQIRGLEMASKNSQDGISLIQTAEGALTETHAILQRMRELTVQAGNGTAAAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVAMAKEMSEFTKNNILSQASQAMLAQANQQPQNVLQLLR

SEQ ID NO: 33 - protein, 237 aa, synthetic construct
SGLRINRAGDDAAGLAISEKMRGQIRGLEMASKNSQDGISLIQTAEGALTETHAILQRMRELTVQAGNGTAAAEDLGAIKDEMDALIEEIDGISNRTEFNGKKLLDGTNSTTFQIAANAGQQLNVKIDSMSSTALGVNALAVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 34 - protein, 237 aa, synthetic construct
SGLRINRAGDDAAGLAISEKMRGQIRGLEMASKNSQDGISLIQTAEGALTETHAILQRMRELTVQAGNGTAAAEDLGAIKDEMDALIEEIDGISNRTEFNGKKLLDGTNSTTFQIAANAGQQLNVKIDSMSSTALGVNALDVTDFAFDDQLKSIDTAINTVSTQRAKLGAVQNRLEHTINNLGASGENLTAAESRIRDVDAAKEMSEFTKNNILSQASQAMLAQANQQPQNVLQLLR

SEQ ID NO: 35 - protein, 275 aa, synthetic construct
MRINHNIAALNTLNRLGSNNGAAQKNMEKLSSGLRINRAGDDAAGLAISEKMRGQIRGLEMASKNSQDGISLIQTAEGALTETHKILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 36 - protein, 275 aa, synthetic construct
MRINHNIAALNTLNRLGSNNGAAQKNMEKLSSGLRINRAGDDAAGLAISEKMRGQIRGLEMASKNSQDGISLIQTAEGALTETHNILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 37 - protein, 275 aa, synthetic construct
MRINHNIAALNTLNRLGSNNGAAQKNMEKLSSGLRINRAGDDAAGLAISEKMRGQIRGLEMASKNSQDGISLIQTAEGALTETHANLQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 38 - protein, 275 aa, synthetic construct
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDARIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 39 - protein, 275 aa, synthetic construct
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLQ

SEQ ID NO: 40 - protein, 237 aa, synthetic construct
SGLRINRAGDDAAGLAISEKMRGQIRGLEMASKNSQDGISLIQTAEGALTETHNILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 41 - protein, 237 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQNVLQLLQ

SEQ ID NO: 42 - protein, 237 aa, synthetic construct
SGLRINRAGDDAAGLAISEKMRGQIRGLEMASKNSQDGISLIQTAEGALTETHNILQRMRELTVQAGNGTQQAEDLGAIKDEMDALIEEIDGISNRTEFNGKKLLDGTNSTTFQIGANAGQQLNVKIDSMSSTALGVNALDVTDFAFDDQLKSIDTAINTVSTQRAKLGAVQNRLEHTINNLGASGENLTAAESRIRDVDMAKEMSEFTKNNILSQASQAMLAQANQQPQNVLQLLQ

SEQ ID NO: 43 - protein, 235 aa, synthetic construct
SGLRINRAGDDAAGLAISEKMRGQIRGLEMASKNSQDGISLIQTAEGALTETHNILQRMRELTVQAGNGTQQAEDLGAIKDEMDALIEEIDGISNRTEFNGKKLLDGTNSTTFQIGANAGQQLNVKIDSMSSTALGVNALDVTAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 44 - protein, 235 aa, synthetic construct
SGLRINRAGDDAAGLAISEKMRGQIRGLEMASKNSQDGISLIQTAEGALTETHAILQRMRELTVQAGNGTQQAEDLGAIKDEMDALIEEIDGISNRTEFNGKKLLDGTNSTTFQIGANAGQQLNVKIDSMSSTALGVNALDVTAFDDQLKSIDTAINTVSTQRAKLGAVQNRLEHTINNLGASGENLTAAESRIRDVDMAKEMSEFTKNNILSQASQAMLAQANQQPQNVLQLLQ

SEQ ID NO: 45 - protein, 235 aa, synthetic construct
SGLRINRAGDDAAGLAISEKMRGQIRGLEMASKNSQDGISLIQTAEGALTETHNILQRMRELTVQAGNGTQQAEDLGAIKDEMDALIEEIDGISNRTEFNGKKLLDGTNSTTFQIGANAGQQLNVKIDSMSSTALGVNALDVTAFDDQLKSIDTAINTVSTQRAKLGAVQNRLEHTINNLGASGENLTAAESRIRDVDMAKEMSEFTKNNILSQASQAMLAQANQQPQNVLQLLQ

(19) United States
(12) Patent Application Publication
(10) Pub. No.: US 2025/0262289 A1
(43) Pub. Date: Aug. 21, 2025

(54) BACILLUS SUBTILIS FLAGELLIN VARIANT AND USE THEREOF

(71) Applicant: MEDISPAN CO., LTD, Seongnam-si (KR)

(72) Inventors: Da Yeon KIM, Seongnam-si Gyeonggi-do (KR); Eun Jae JEON, Seongnam-si Gyeonggi-do (KR); Kye Young KIM, Seongnam-si Gyeonggi-do (KR); Sun Mi KANG, Seongnam-si Gyeonggi-do (KR)

(21) Appl. No.: 19/049,517

(22) Filed: Feb. 10, 2025

Related U.S. Application Data
(63) Continuation-in-part of application No. PCT/KR2023/011794, filed on Aug. 9, 2023.

(30) Foreign Application Priority Data
Aug. 10, 2022 (KR) 10-2022-0100229

(51) Int. Cl. A61K 39/07; A61K 35/74; A61K 39/00; C07K 14/32

(52) U.S. Cl. CPC A61K 39/07; C07K 14/32; A61K 35/74; A61K 2039/522

(57) ABSTRACT

The present invention relates to a Bacillus subtilis flagellin variant and a use thereof, and more specifically to a flagellin variant and a use thereof, the flagellin variant having improved storage stability and reduced immunogenicity compared to flagellin derived from wild-type Bacillus subtilis.

### CROSS-REFERENCE TO RELATED APPLICATIONS

This application is a continuation-in-part of PCT Application No. PCT/KR2023/011794 filed Aug. 9, 2023, which claims the benefit of priority to Korean Application No. 10-2022-0100229 filed Aug. 10, 2022, each of which is incorporated herein by reference in their entirety.

### REFERENCE TO SEQUENCE LISTING

The sequence listing submitted on Feb. 10, 2025, as an .XML file entitled “11239-026US1 2025_02_10 Sequence Listing” created on Aug. 9, 2023, and having a file size of 50,137 bytes is hereby incorporated by reference pursuant to 37 C.F.R. § 1.52 (e) (5).

### TECHNICAL FIELD

This application claims priority to Korean Patent Application No. 10-2022-0100229, filed on Aug. 10, 2022, the entire contents of which are hereby incorporated by reference.

The present invention relates to a Bacillus subtilis flagellin variant and use thereof, and more specifically, to a flagellin variant derived from wild-type Bacillus subtilis that have improved storage stability and reduced immunogenicity compared to the wild-type flagellin, and its uses.

### BACKGROUND OF THE INVENTION

Flagella are crucial components that determine the motility of bacteria and are largely composed of a hook, a basal body, and a filament. It is known that flagella determine bacterial swimming or swarming motility, bacterial taxis, and form biofilms, which in turn determine the adhesion ability of pathogenic microorganisms. The structural unit protein that constitutes the filament of the flagella is called flagellin, and flagellin assembles regularly to form the filament. Hayashi et al. reported that TLR5 expressed in mammals recognizes flagellin from both Gram-negative and Gram-positive bacteria and activates NF-κB (Hayashi F, Smith KD, Ozinsky A, Hawn T R, Yi E C, Goodlett D R, Eng J K, Akira S, Underhill D M, Aderem A: Nature 410:1099-1103, 2001). Flagellin is a structural protein that assembles into the whip-like filament of bacterial flagella, extending from the cell surface to enable bacterial movement. Flagellin acts as a virulence factor in pathogenic bacteria, promoting invasion and penetration into host cells. Since flagellin is exclusively found in bacteria and is one of the most abundant proteins in flagellated bacteria, it becomes a primary target for host immune surveillance. Upon bacterial invasion, flagellin is detected by Toll-like receptor 5 (TLR5) and NAIP5/NLRC4 in the host, activating innate immunity that contributes to the immediate elimination of pathogens.

TLR5 is an innate immune receptor located on the cell surface, consisting of an extracellular leucine-rich repeat (LRR) domain, a transmembrane domain, and an intracellular domain. TLR5 recognizes flagellin as a pathogen-associated molecular pattern using its extracellular domain and activates the MyD88-dependent signaling pathway and NF-κB-mediated inflammatory cytokine production.

Flagellin has garnered interest as a target for developing vaccine carrier proteins or adjuvants because it serves as the first line of defense against flagellated pathogenic bacteria. Fusion proteins of antigens and flagellin have been proven effective as experimental vaccines against various infectious diseases, including West Nile fever, malaria, plague, and tuberculosis. TLR5 activation by flagellin has also been reported to protect hematopoietic cells and gastrointestinal tissues from radiation and affect the survival and growth of cancer cells. Additionally, flagellin has been reported to exhibit therapeutic effects in diseases such as liver disease, metabolic syndrome, and colitis.

Meanwhile, immune responses to biological therapeutics can be widely induced against both non-human and human-derived proteins. These responses can weaken clinical efficacy, limit potency, and sometimes cause pathological conditions or even patient death. Such immune responses are often triggered by antibody reactions. The patient's antibody response depends on the presence of B-cell epitopes and T-cell epitopes. When B-cell receptors in the body recognize and bind to administered foreign proteins, the foreign proteins are internalized into B-cells through receptor-mediated endocytosis and undergo protein hydrolysis. The resulting peptides are then presented by MHC class II molecules. When T-helper cells recognize T-cell epitopes, they stimulate corresponding B-cells to proliferate and differentiate into plasma cells that produce antibodies. To address the reduction in in vivo utility due to the immunogenicity of foreign proteins, various de-immunization techniques have been provided in the past. However, no technology has been reported to date for reducing or eliminating the immunogenicity of flagellin.

### Problem to be Solved

The present inventors have conducted extensive research to develop a variant protein of Bacillus subtilis flagellin that maintains its physiological activity while reducing immunogenicity in the body, thereby minimizing side effects and improving storage stability. As a result, the present invention has been completed.

Therefore, an object of the present invention is to provide a Bacillus subtilis flagellin variant comprising an amino acid sequence having at least 50% identity to a wild-type Bacillus subtilis flagellin of SEQ ID NO: 1 and having reduced immunogenicity compared to the wild-type Bacillus subtilis flagellin.

Another object of the present invention is to provide a polynucleotide comprising a nucleotide sequence encoding the Bacillus subtilis flagellin variant, a vector comprising the polynucleotide, and a host cell transformed with the vector.

Another object of the present invention is to provide a pharmaceutical composition for preventing or treating damage caused by radiation exposure; for preventing or treating reperfusion injury; for preventing or treating inflammatory bowel disease; for preventing or treating autoimmune disease; for preventing or treating viral infection; for preventing or treating metabolic disease; for preventing or treating aging; for enhancing immune function; for preventing or treating cancer; for preventing or treating liver disease; or for preventing or treating colitis, comprising the Bacillus subtilis flagellin variant as an active ingredient.

Another object of the present invention is to provide a pharmaceutical composition for preventing or treating damage caused by radiation exposure; for preventing or treating reperfusion injury; for preventing or treating inflammatory bowel disease; for preventing or treating autoimmune disease; for preventing or treating viral infection; for preventing or treating metabolic disease; for preventing or treating aging; for enhancing immune function; for preventing or treating cancer; for preventing or treating liver disease; or for preventing or treating colitis, consisting of the Bacillus subtilis flagellin variant.

Another object of the present invention is to provide a pharmaceutical composition for preventing or treating damage caused by radiation exposure; for preventing or treating reperfusion injury; for preventing or treating inflammatory bowel disease; for preventing or treating autoimmune disease; for preventing or treating viral infection; for preventing or treating metabolic disease; for preventing or treating aging; for enhancing immune function; for preventing or treating cancer; for preventing or treating liver disease; or for preventing or treating colitis, consisting essentially of the Bacillus subtilis flagellin variant.

Another object of the present invention is to provide a vaccine adjuvant comprising the Bacillus subtilis flagellin variant as an active ingredient.

Another object of the present invention is to provide a use of the Bacillus subtilis flagellin variant according to any one of claims 1 to 13 for manufacturing a pharmaceutical composition for treating damage caused by radiation exposure; for treating reperfusion injury; for treating inflammatory bowel disease; for treating autoimmune disease; for treating viral infection; for treating metabolic disease; for treating aging; for enhancing immune function; for treating cancer; for treating liver disease; or for treating colitis.

Another object of the present invention is to provide a method for treating damage caused by radiation exposure; treating reperfusion injury; treating inflammatory bowel disease; treating autoimmune disease; treating viral infection; treating metabolic disease; treating aging; enhancing immune function; treating cancer; treating liver disease; or treating colitis, comprising administering an effective amount of a pharmaceutical composition comprising the Bacillus subtilis flagellin variant according to any one of claims 1 to 13 as an active ingredient to a subject in need thereof.

### Means for Solving the Problem

To achieve the aforementioned objective of the present invention, the present invention provides a Bacillus subtilis flagellin variant comprising an amino acid sequence having at least 50% identity to a wild-type Bacillus subtilis flagellin of SEQ ID NO: 1 and having reduced immunogenicity compared to the wild-type Bacillus subtilis flagellin.

To achieve another objective of the present invention, the present invention provides a polynucleotide comprising a nucleotide sequence encoding the Bacillus subtilis flagellin variant, a vector comprising the polynucleotide, and a host cell transformed with the vector.

To achieve another objective of the present invention, the present invention provides a pharmaceutical composition for preventing or treating damage caused by radiation exposure; for preventing or treating reperfusion injury; for preventing or treating inflammatory bowel disease; for preventing or treating autoimmune disease; for preventing or treating viral infection; for preventing or treating metabolic disease; for preventing or treating aging; for enhancing immune function; for preventing or treating cancer; for preventing or treating liver disease; or for preventing or treating colitis, comprising the Bacillus subtilis flagellin variant as an active ingredient.

Additionally, to achieve another objective of the present invention, the present invention provides a pharmaceutical composition for preventing or treating damage caused by radiation exposure; for preventing or treating reperfusion injury; for preventing or treating inflammatory bowel disease; for preventing or treating autoimmune disease; for preventing or treating viral infection; for preventing or treating metabolic disease; for preventing or treating aging; for enhancing immune function; for preventing or treating cancer; for preventing or treating liver disease; or for preventing or treating colitis, consisting of the Bacillus subtilis flagellin variant.

Additionally, to achieve another objective of the present invention, the present invention provides a pharmaceutical composition for preventing or treating damage caused by radiation exposure; for preventing or treating reperfusion injury; for preventing or treating inflammatory bowel disease; for preventing or treating autoimmune disease; for preventing or treating viral infection; for preventing or treating metabolic disease; for preventing or treating aging; for enhancing immune function; for preventing or treating cancer; for preventing or treating liver disease; or for preventing or treating colitis, consisting essentially of the Bacillus subtilis flagellin variant.

To achieve another objective of the present invention, the present invention provides a vaccine adjuvant comprising the Bacillus subtilis flagellin variant as an active ingredient.

To achieve another objective of the present invention, the present invention provides a use of the Bacillus subtilis flagellin variant according to any one of claims 1 to 13 for manufacturing a pharmaceutical composition for treating damage caused by radiation exposure; for treating reperfusion injury; for treating inflammatory bowel disease; for treating autoimmune disease; for treating viral infection; for treating metabolic disease; for treating aging; for enhancing immune function; for treating cancer; for treating liver disease; or for treating colitis.

To achieve another objective of the present invention, the present invention provides a method for treating damage caused by radiation exposure; treating reperfusion injury; treating inflammatory bowel disease; treating autoimmune disease; treating viral infection; treating metabolic disease; treating aging; enhancing immune function; treating cancer; treating liver disease; or treating colitis, comprising administering an effective amount of a pharmaceutical composition comprising the Bacillus subtilis flagellin variant according to any one of claims 1 to 13 as an active ingredient to a subject in need thereof.

The single-letter (three-letter) abbreviations of amino acids used in this specification refer to the following amino acids according to the standard abbreviations in the field of biochemistry: A (Ala): alanine; C (Cys): cysteine; D (Asp): aspartic acid; E (Glu): glutamic acid; F (Phe): phenylalanine; G (Gly): glycine; H (His): histidine; I (Ile): isoleucine; K (Lys): lysine; L (Leu): leucine; M (Met): methionine; N (Asn): asparagine; O (Ply): pyrrolysine; P (Pro): proline; Q (Gln): glutamine; R (Arg): arginine; S (Ser): serine; T (Thr): threonine; U (Sec): selenocysteine; V (Val): valine; W (Trp): tryptophan; Y (Tyr): tyrosine.

The notation “(amino acid single-letter) (amino acid position) (amino acid single-letter)” used in this specification means that the amino acid at the corresponding position of the wild-type protein is substituted with the amino acid indicated by the subsequent notation. For example, R57H indicates that the arginine at the 57th position of the wild-type protein is substituted with histidine. Hereinafter, the present invention will be described in detail.

The present invention provides a Bacillus subtilis flagellin variant comprising an amino acid sequence having at least 50% identity to a wild-type Bacillus subtilis flagellin of SEQ ID NO: 1 and having reduced immunogenicity compared to the wild-type Bacillus subtilis flagellin.

In the present invention, the flagellin can induce an immune response in the infected host when a flagellated bacterium is infected. More specifically, Toll-like receptor 5 (TLR5) present on the surface of human cell membranes interacts with the flagellin to induce intracellular signal transduction, thereby increasing the expression of the transcription factor NF-kB, which not only induces innate immune signal activation but also regulates the acquired immune response.

In the present invention, SEQ ID NO: 1 is a wild-type flagellin derived from B. subtilis (Bsflagellin).

[SEQ ID NO: 1]
MRINHNIAALNTLNRLGSNNGAAQKNMEKLSSGLRINRAGDDAAGLAISE
KMRGQIRGLEMASKNSQDGISLIQTAEGALTETHAILQRMRELTVQAGNT
GTQQAEDLGAIKDEMDALIEEIDGISNRTEFNGKKLLDGTNSTDGFTFQI
GANAGQQLNVKIDSMSSTALGVNALDVTDFAATAFDDQLKSIDTAINTVS
TQRAKLGAVQNRLEHTINNLGASGENLTAAESRIRDVDMAKEMSEFTKNN
ILSQASQAMLAQANQQPQNVLQLLR

In one aspect of the present invention, the Bsflagellin variant may comprise an amino acid sequence having at least 50%, 55%, 60%, 65%, 70%, 75%, 80%, 85%, 90%, 91%, 92%, 93%, 94%, 95%, 96%, 97%, 98%, or 99% identity to the wild-type Bsflagellin of SEQ ID NO: 1. The amino acid sequence having the sequence identity may include one or more substitutions, deletions, additions, and/or insertions as arbitrary changes in the amino acid sequence of SEQ ID NO: 1. Such variants may occur naturally or may be synthetically generated by modifying or altering one or more of the peptide sequences of the present invention using any of a number of techniques well known in the art and evaluating their biological activity as described herein.

In one embodiment of the present invention, the amino acid sequence having at least 50% sequence identity to SEQ ID NO: 1 includes conservative substitutions. ‘Conservative substitution’ refers to a substitution where one amino acid is replaced with another amino acid having similar properties, such that a person skilled in the art would predict that the secondary structure and hydropathic nature (hydrophobic or hydrophilic properties) of the peptide would remain substantially unchanged. Generally, the following groups of amino acids exhibit conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

The modification may be performed within the structure of the wild-type Bsflagellin according to SEQ ID NO: 1 to obtain a functional molecule encoding a protein variant or derivative with desired (preferred) characteristics. When it is desired to alter the amino acid sequence of the peptide to produce an equivalent or improved variant of Bsflagellin according to SEQ ID NO: 1, a person skilled in the art can change one or more codons based on the protein codon information known in the art.

For instance, specific amino acids can be substituted with other amino acids within a protein or peptide structure without significant loss of interactive binding capability, such as the structure of a receptor, antigen-binding site of an antibody, or binding site on a substrate molecule. This is because of the interactive capability and properties of a protein, as generally defined biological functional activity. Specific amino acid sequence substitutions can take place within the protein or peptide sequence, and of course, within the underlying DNA coding sequence, while still a protein with the same or similar properties ca be obtained.

Therefore, various changes in the DNA sequence encoding Bsflagellin according to SEQ ID NO: 1 are considered without significant loss of the desired utility or activity. These modifications may also take into account the hydropathic (hydrophobic or hydrophilic properties) index of amino acids. The importance of the hydropathic amino acid index, which imparts interactive biological function to proteins, is generally understood in the art (see Kyte and Doolittle, 1982, incorporated herein by reference). For example, it is known that the relative hydropathic properties of amino acids contribute to the secondary structure of the resulting protein, which in turn defines the interaction of the protein with other molecules, such as enzymes, substrates, receptors, DNA, antibodies, antigens, etc. Each amino acid is assigned a hydropathic index based on its hydrophobicity and charge characteristics (Kyte and Doolittle, 1982). These values are as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5).

It is known in the art that specific amino acids can be substituted by other amino acids with similar hydropathic indices or scores, thus a protein with similar biological activity can be obtained (i.e., a biologically functionally equivalent protein can still be obtained). In such modifications, amino acids with a hydropathic index within ±2 are preferred for substitution, those within ±1 are particularly preferred, and those within ±0.5 are even more particularly preferred.

It is also understood in the art that substitutions of the same amino acid can be effectively performed based on hydrophilicity. As described in U.S. Pat. No. 4,554,101, the following hydrophilicity values are assigned to amino acid residues: arginine (+3.0); lysine (+3.0); aspartic acid (+3.0=1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that amino acids can be substituted by other amino acids with similar hydrophilicity values, thereby yielding a biologically equivalent protein. In such modifications, substitutions of amino acids with hydrophilicity values within ±2 are preferred, those within ±1 are particularly preferred, and those within ±0.5 are even more particularly preferred.

As outlined above, amino acid substitutions can be based on the relative similarity of the side-chain substituents of the amino acids, such as their hydrophobicity, hydrophilicity, charge, size, etc. Exemplary substitutions considering the various features described above are well known to those skilled in the art and include: arginine and lysine; glutamic acid and aspartic acid; serine and threonine; glutamine and asparagine; valine, leucine, and isoleucine.

Amino acid substitutions can also be performed based on the similarity of the residues' polarity, charge, solubility, hydrophobicity, hydrophilicity, and/or amphipathic properties. For example, negatively charged amino acids include aspartic acid and glutamic acid; positively charged amino acids include lysine and arginine; and amino acids with non-charged polar head groups with similar hydrophilicity values include leucine, isoleucine, and valine; glycine and alanine; asparagine and glutamine; serine, threonine, phenylalanine, and tyrosine.

Additionally, proteins with more than 50% sequence identity to Bsflagellin according to SEQ ID NO: 1 may include non-conservative changes in amino acids. Furthermore, for example, they can be modified by deletions or additions of amino acids that have minimal impact on the secondary structure and hydropathic properties of Bsflagellin according to SEQ ID NO: 1.

When comparing amino acid sequences of proteins, if the sequences of amino acids in two sequences are identical when the two sequences are aligned for maximum correspondence as described below, the two sequences are said to be “identical.” Comparison between two sequences is typically performed by identifying and comparing local regions of sequence similarity by comparing alignments on a comparison window. As used herein, a “comparison window” refers to a segment of at least about 20 contiguous positions, typically 30 to 75, 40 to 50 contiguous positions, in which the sequences can be compared to a reference sequence of the same number of contiguous positions after optimal alignment of the two sequences.

Optimal alignment of sequences for comparison can be performed using, for example, the Megalign program in the Lasergene suite of bioinformatics software (DNASTAR, Inc., Madison, Wis.) using default parameters. This program includes several alignment schemes described in the following references: Dayhoff, M. O. (1978) A model of evolutionary change in proteins-Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Nat'l Acad., Sci. USA 80:726-730.

Alternatively, optimal alignment of sequences for comparison can be performed by the partial identity algorithm of Smith and Waterman (1981) Add.APL.Math 2:482, the identity alignment algorithm of Needleman and Wunsch (1970) J.Mol.Biol.48:443, the similarity search method of Pearson and Lipman (1988) Proc.Natl.Acad.Sci.USA 85:2444, and computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

An example of a suitable algorithm for determining sequence identity and sequence similarity percentages (percent) may be the BLAST and BLAST2.0 algorithms, which are described in Altschul et al. (1977) Nucl. Acids Res. 25:3389-3402 and Altschul et al. (1990) J. Mol. Biol. 215:403-410, respectively. BLAST and BLAST2.0 can be used to determine the sequence identity percentage for the polynucleotides and polypeptides of the present invention, for example, using the parameters described in the specification. Software for performing BLAST analysis is publicly available through the National Center for Biotechnology Information. For amino acid sequences, a scoring matrix can be used to calculate the cumulative score.

The extension of word hits in each direction stops under the following conditions: if the cumulative alignment score is decreased by quantity X from its maximum achieved value, if the cumulative score becomes zero or less due to accumulation of the alignment of one or more negatively scoring residues, or if the end of any sequence is reached. The BLAST algorithm parameters W, T, and X determine the sensitivity and speed of the alignment.

In one exemplary approach, the ‘percentage of sequence identity’ is determined by comparing two optimal aligned sequences in a comparison window of at least 20 positions, where the domain of the polypeptide sequence in the comparison window may include up to 20 percent, typically 5-15 percent, or 10-12 percent additions or deletions (i.e., gaps) compared to the reference sequence (which does not include additions or deletions). The percentage is calculated by determining the number of positions with identical amino acid residues in both sequences thereby obtaining the number of matching positions, dividing the number of matching positions by the total number of positions in the reference sequence (i.e., window size), and multiplying the result by 100 to calculate the sequence identity percentage.

The term “percent (%) sequence identity” as used in the present invention is defined as the percentage of amino acid residues in the candidate sequence that are identical to the amino acid residues in the reference protein, after aligning the sequences and introducing gaps, without considering any conservative substitutions as part of the sequence identity if necessary to achieve the maximum percent sequence identity. Alignments for determining percent amino acid identity can be achieved utilizing any publicly available computer softwares and using various methods within the skill of the art, for example, methods described in reference [Current Protocols in Molecular Biology (Ausubel et al., eds., 1987)], and using software such as BLAST, BLAST-2, ALIGN, or Megalign (DNASTAR). A person skilled in the art can determine appropriate parameters for alignment measurement, including any algorithm necessary to achieve maximum alignment over the entire length of the sequences being compared. For the purposes of this specification, the percent (%) amino acid sequence identity of a given amino acid sequence A to a given amino acid sequence B is calculated as follows: 100 times the fraction X/Y, where X is the number of identical amino acid residue scores in the sequence alignment program's alignment of A and B, and Y is the total number of amino acid residues in B. It is understood that if the length of amino acid sequence A is not the same as the length of amino acid sequence B, the percent (%) amino acid sequence identity of A to B is not the same as the percent (%) amino acid sequence identity of B to A.

In certain embodiments, chemoselective ligation techniques can be used to produce a protein having at least 50% sequence identity to Bsflagellin of SEQ ID NO: 1, for example, by attaching a polymer in a site-specific and controlled manner. These techniques typically rely on the incorporation of a chemoselective anchor into the protein backbone by chemical or recombinant means, followed by subsequent modification with a polymer carrying a complementary linker. As a result, the assemble process and the ensuing covalent structure of the assembled protein-polymer conjugate is controlled, allowing for the rational optimization of drug properties such as efficacy and pharmacokinetic characteristics. For example, allowing selective attachment of PEG improves their pharmacokinetic properties.

In one aspect of the present invention, the Bsflagellin variant may have reduced immunogenicity by at least 4%, 5%, 10%, 15%, 20%, 25%, 30%, 40%, 45%, 50%, 55%, 60%, 65%, 70%, 75%, 80%, 85%, or 90% compared to wild-type Bsflagellin.

In one aspect of the present invention, the term “immunogenicity” refers to the ability of a specific substance triggering immune responses to induce a humoral and/or cell-mediated immune response in an animal, such as a human. Such immunogenicity can inactivate therapeutic effects and cause side effects. In a preferred aspect of the present invention, the immunogenicity may refer to the activation of B-cells due to the presence of B-cell epitopes recognized by B-cells or antibodies secreted by B-cells.

In another aspect of the present invention, the Bsflagellin variant may be characterized by improved productivity compared to wild-type Bsflagellin. In one aspect of the present invention, the productivity may refer to the protein productivity in host cells transformed with a vector according to conventional recombinant protein production methods. Preferably, under the same conditions, the Bsflagellin variant may be characterized by a productivity increase of 50%, 100%, 150%, or 200% or more compared to wild-type Bsflagellin.

In another aspect of the present invention, the Bsflagellin variant may be characterized by an increased residual rate of the protein after storage for 2 weeks at 37° C. compared to wild-type Bsflagellin. Preferably, the Bsflagellin may be characterized by an increase in the residual rate of the protein by at least 50%, 55%, 60%, 65%, 70%, 75%, 80%, 85%, 90%, 95%, or 100% or more after storage for 2 weeks at 37° C. compared to wild-type Bsflagellin.

In one aspect of the present invention, the Bsflagellin variant having at least 50% sequence identity to the flagellin of SEQ ID NO: 1 may activate the TLR5 pathway at 50%, 60%, 70%, 80%, 90%, 95%, or 100% levels of wild-type Bsflagellin, or may activate the TLR5 pathway to the same or essentially the same extent as wild-type Bsflagellin or Bsflagellin fragments, or may activate the TLR5 pathway to a higher extent compared to wild-type Bsflagellin or Bsflagellin fragments.

The Bsflagellin variant according to the present invention is characterized by providing additional advantages as low immunogenicity, improved productivity, structural stability, pharmacokinetic advantages, low in vivo toxicity, and storage stability, while exhibiting equal or enhanced ability to activate TLR5 and NF-kB signaling activity compared to wild-type Bsflagellin of SEQ ID NO: 1.

In one aspect of the present invention, the Bsflagellin variant may be characterized by comprising one or more mutations from the following (1) to (7) in the wild-type Bsflagellin sequence of SEQ ID NO: 1:
(1) an amino acid substitution at one or more positions selected from the group consisting of L34, S49, M52, L59, A85, 186, Q103, Q104, L118, G151, G155, D176, G224, E231, D236, D238, M239 and R275;
(2) A deletion of the amino-terminal 0 domain (ND0, amino acids 1-31);
(3) A deletion of one or more amino acids within the amino-terminal 1 domain (ND1, amino acids 43-181);
(4) A deletion of one or more amino acids within the ND0-ND1 interface region (amino acids 32-42);
(5) A deletion of one or more amino acids within the carboxy-terminal 1 domain (CD1, amino acids 182-235);
(6) A deletion of one or more amino acids within the carboxy-terminal 0 domain (CD0, amino acids 239-275); and
(7) A deletion of the CD1-CD0 interface region (amino acids 236-238).

In one aspect of the present invention, the amino acid substitution of (1) may be characterized by being one or more selected from the group consisting of L34C, S49C, M52C, L59C, A85K, A85N, I86N, Q103A, Q104A, L118R, G151A, G155A, D176A, G224C, E231C, D236C, D238A, M239A and R275Q.

In one aspect of the present invention, the deletion of one or more amino acids within ND1 of (3) may be characterized by being one or more deletions selected from the group consisting of a deletion of 43, a deletion of 43-50, a deletion of 100, a deletion of 181, a deletion of 144-146, a deletion of 164-179, and a deletion of 152-181 in the amino acid sequence of the wild-type Bacillus subtilis flagellin of SEQ ID NO: 1.

In one aspect of the present invention, the deletion of one or more amino acids within the ND0-ND1 interface region of (4) may be characterized by being one deletion selected from the group consisting of a deletion of 32-33, a deletion of 32-41, and a deletion of 32-42 in the amino acid sequence of the wild-type Bacillus subtilis flagellin of SEQ ID NO: 1.

In one aspect of the present invention, the deletion of one or more amino acids within CD1 of (5) may be characterized by being one or more deletions selected from the group consisting of a deletion of 181-183, a deletion of 182-186, and a deletion of 230-235.

In one aspect of the present invention, the deletion of one or more amino acids within CD0 of (6) may be characterized by being one or more deletions selected from the group consisting of a deletion of 266-275, a deletion of 270-275, a deletion of 239-253, a deletion of 243-253, a deletion of 243-260, and a deletion of 239-275.

In a preferred aspect of the present invention, the variant may be characterized by comprising the mutations of (2), (3), and (5), and may or may not comprise the mutation of (1).

In a preferred aspect of the present invention, the variant may be characterized by comprising a deletion of the amino-terminal 0 domain (ND0, amino acids 1-31); and deletions of T100, D144, G145, F146, A181, A182, and T183; and may or may not comprise one or more amino acid substitutions at positions selected from the group consisting of L34, S49, M52, L59, A85, 186, Q103, Q104, L118, G151, G155, D176, G224, E231, D236, D238, M239 and R275 in the wild-type Bsflagellin of SEQ ID NO: 1.

In a preferred aspect of the present invention, the variant may be characterized by comprising a deletion of the amino-terminal 0 domain (ND0, amino acids 1-31); and deletions of T100, D144, G145, F146, A181, A182, and T183; and may or may not comprise one or more amino acid substitutions selected from the group consisting of L34C, S49C, M52C, L59C, A85K, A85N, 186N, Q103A, Q104A, L118R, G151A, G155A, D176A, G224C, E231C, D236C, D238A, M239A and R275Q in the wild-type Bsflagellin of SEQ ID NO: 1.

In another aspect of the present invention, the variant may be characterized by comprising the mutations of (2), (3), and (5), and may or may not comprise the mutation of (1) in the wild-type Bsflagellin of SEQ ID NO: 1.

In a preferred aspect of the present invention, the variant may be characterized by comprising a deletion of the amino-terminal 0 domain (ND0, amino acids 1-31); and deletions of T100, D144, G145, F146, D179, F180, A181, A182, and T183; and may or may not comprise one or more amino acid substitutions at positions selected from the group consisting of L34, S49, M52, L59, A85, 186, Q103, Q104, L118, G151, G155, D176, G224, E231, D236, D238, M239 and R275 in the wild-type Bsflagellin of SEQ ID NO: 1.

In a preferred aspect of the present invention, the variant may be characterized by comprising a deletion of the amino-terminal 0 domain (ND0, amino acids 1-31); and deletions of T100, D144, G145, F146, D179, F180, A181, A182, and T183; and may or may not comprise one or more amino acid substitutions selected from the group consisting of L34C, S49C, M52C, L59C, A85K, A85N, I86N, Q103A, Q104A, L118R, G151A, G155A, D176A, G224C, E231C, D236C, D238A, M239A, and R275Q in the wild-type Bsflagellin of SEQ ID NO: 1.

In one aspect of the present invention, the Bacillus subtilis flagellin variant may be characterized by being selected from the following variants:

The Bacillus subtilis flagellin variant may be characterized by being selected from the following variants:
A85K, A85N, 186N, L118R, or R275Q in SEQ ID NO: 1;
a deletion of 1-31 in SEQ ID NO: 1;
deletions of T100, D144, G145, F146, A181, A182, and T183 in SEQ ID NO: 1;
deletions of 1-31, T100, D144, G145, F146, A181, A182, and T183 in SEQ ID NO: 1;
deletions of 1-31, T100, D144, G145, F146, D179, F180, A181, A182, and T183 in SEQ ID NO: 1;
deletions of 1-33, T100, D144, G145, F146, A181, A182, and T183 in SEQ ID NO: 1;
deletions of 1-41, T100, D144, G145, F146, A181, A182, and T183 in SEQ ID NO: 1;
deletions of 1-43, T100, D144, G145, F146, A181, A182, and T183 in SEQ ID NO: 1;
deletions of 1-50, T100, D144, G145, F146, A181, A182, and T183 in SEQ ID NO: 1;
deletions of 1-31, 164-179, T100, D144, G145, F146, A181, A182, and T183 in SEQ ID NO: 1;
deletions of 1-31, 152-186, T100, D144, G145, and F146 in SEQ ID NO: 1;
deletions of 1-31, 270-275, T100, D144, G145, F146, A181, A182, and T183 in SEQ ID NO: 1;
deletions of 1-31, 266-275, T100, D144, G145, F146, A181, A182, and T183 in SEQ ID NO: 1;
deletions of 1-31, 236-275, T100, D144, G145, F146, A181, A182, and T183 in SEQ ID NO: 1;
deletions of 1-31, 230-275, T100, D144, G145, F146, A181, A182, and T183 in SEQ ID NO: 1;
deletions of 1-50, 236-275, T100, D144, G145, F146, A181, A182, and T183 in SEQ ID NO: 1;
deletions of 1-31, 243-253, T100, D144, G145, F146, A181, A182, and T183 in SEQ ID NO: 1;
deletions of 1-31, 239-253, T100, D144, G145, F146, A181, A182, and T183 in SEQ ID NO: 1;
deletions of 1-31, 243-260, T100, D144, G145, F146, A181, A182, and T183 in SEQ ID NO: 1;
deletions of 1-31, 236-253, T100, D144, G145, F146, A181, A182 and T183 in SEQ ID NO: 1;
deletions of 1-31, T100, D144, G145, F146, A181, A182, and T183, and L34C and D236C in SEQ ID NO: 1;
deletions of 1-31, T100, D144, G145, F146, A181, A182, and T183, and S49C and E231C in SEQ ID NO: 1;
deletions of 1-31, T100, D144, G145, F146, A181, A182, and T183, and M52C and E231C in SEQ ID NO: 1;
deletions of 1-31, T100, D144, G145, F146, A181, A182, and T183, and L59C and G224C in SEQ ID NO: 1;
deletions of 1-31, T100, D144, G145, F146, A181, A182, and T183, and Q103A and Q104A in SEQ ID NO: 1;
deletions of 1-31, T100, D144, G145, F146, A181, A182, and T183, and G151A in SEQ ID NO: 1;
deletions of 1-31, T100, D144, G145, F146, A181, A182, and T183, and G155A in SEQ ID NO: 1;
deletions of 1-31, T100, D144, G145, F146, A181, A182, and T183, and D176A in SEQ ID NO: 1;
deletions of 1-31, T100, D144, G145, F146, A181, A182, and T183, and D238A in SEQ ID NO: 1;
deletions of 1-31, T100, D144, G145, F146, A181, A182, and T183, and Q103A, Q104A, and G155A in SEQ ID NO: 1;
deletions of 1-31, T100, D144, G145, F146, A181, A182, and T183, and Q103A, Q104A, and D176A in SEQ ID NO: 1;
deletions of 1-31, T100, D144, G145, F146, A181, A182, and T183, and Q103A, Q104A, and D238A in SEQ ID NO: 1;
deletions of 1-31, T100, D144, G145, F146, A181, A182, and T183, and Q103A, Q104A, G151A, and D176A in SEQ ID NO: 1;
deletions of 1-31, T100, D144, G145, F146, A181, A182, and T183, and Q103A, Q104A, G151A, and M239A in SEQ ID NO: 1;
deletions of 1-31, T100, D144, G145, F146, A181, A182, and T183, and A85N in SEQ ID NO: 1;
deletions of 1-31, T100, D144, G145, F146, A181, A182, and T183, and R275Q in SEQ ID NO:

1;
deletions of 1-31, T100, D144, G145, F146, A181, A182, and T183, and A85N and R275Q in SEQ ID NO: 1;
deletions of 1-31, T100, D144, G145, F146, D179, F180, A181, A182, and T183, and A85N in SEQ ID NO: 1;
deletions of 1-31, T100, D144, G145, F146, D179, F180, A181, A182, and T183, and R275Q in SEQ ID NO: 1; and
deletions of 1-31, T100, D144, G145, F146, D179, F180, A181, A182, and T183, and A85N and R275Q in SEQ ID NO: 1.

In one aspect of the present invention, the flagellin variant may be characterized by retaining NF-kB signaling activity.

In one aspect of the present invention, the flagellin variant may comprise a tag. In another aspect, the tag is attached to the N-terminus of the flagellin variant. In yet another aspect, the tag is attached to the C-terminus of the flagellin variant.

The flagellin variant may comprise a signal (or leader) sequence at the N-terminus of the protein, which directs the transport of the protein either during or after translation. The flagellin variant may also be conjugated with linker sequences or other sequences to facilitate protein synthesis, purification, or identification (e.g., polyHis), or to enhance binding to a solid support.

The flagellin variant of the present invention may comprise a pharmaceutically acceptable salt form. Examples of the pharmaceutically acceptable salts include, but are not limited to, hydrochloride, sulfate, phosphate, acetate, citrate, tartrate, succinate, lactate, maleate, fumarate, oxalate, methanesulfonate, or p-toluenesulfonate.

In some cases, the flagellin variant of the present invention may be a full-length flagellin or a fragment thereof modified by phosphorylation, sulfation, acrylation, glycosylation, methylation, farnesylation, etc.

In the present invention, the flagellin variant may be in the form of a fusion protein comprising other polypeptides. For example, the flagellin variant may be a fusion protein comprising one or more antigens. Non-limiting examples of such antigens include S. pneumoniae PspA1 antigen, S. pneumoniae PspA2 antigen, S. pneumoniae PspA3 antigen, S. pneumoniae PspA4 antigen, S. pneumoniae PspA5 antigen, and/or S. pneumoniae PspA6 antigen. Alternatively, the flagellin variant may be in the form of a fusion protein conjugated with one or more immunomodulatory substances for instance. The immunomodulatory substances may include, without limitation, those known in the art to enhance immune responses, such as interferon-α, interferon-β, interferon-γ, interferon-ω, interferon-τ, interleukin-1α, interleukin-1β, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-18, B cell growth factor, CD40 ligand, TNF-α, TNF-β, CCL25, CCL28, or active fragments thereof.

The present invention also provides a polynucleotide comprising a nucleotide sequence encoding the Bacillus subtilis flagellin variant.

The polynucleotide is not particularly limited in terms of base combination as long as it comprises a nucleotide sequence encoding the polypeptide of the present invention. The polynucleotide may be provided in the form of a single-stranded or double-stranded nucleic acid molecule including DNA, cDNA, or RNA sequence.

The present invention also provides a vector comprising the polynucleotide.

The vector of the present invention may include, but is not limited to, plasmid vectors, cosmid vectors, bacteriophage vectors, and viral vectors. The vector of the present invention may be a conventional cloning vector or an expression vector, and the expression vector may comprise expression control sequences such as promoters, operators, initiation codons, termination codons, polyadenylation signals, and enhancers, as well as signal sequences or leader sequences for membrane targeting or secretion, and may be variously constructed according to the purpose. The polynucleotide sequence according to the present invention may be operably linked to the expression control sequences, and the operably linked gene sequence and expression control sequences may be included in a single expression vector comprising a selection marker and a replication origin. “Operably linked” means that the sequences are connected in such a way that the expression of the gene is enabled when the appropriate molecules bind to the expression control sequences, implying that one nucleic acid fragment is linked to another nucleic acid fragment so that its function or expression is influenced by the other nucleic acid fragment. “Expression control sequence” refers to a DNA sequence that regulates the expression of an operably linked polynucleotide sequence in a specific host cell. Such control sequences include promoters for transcription, any operator sequences for regulating transcription, sequences coding for appropriate mRNA ribosome binding sites, and sequences regulating the termination of transcription and translation. The vector may also include a selection marker for selecting host cells containing the vector, and in the case of a replicable vector, it includes an origin of replication. The present invention also provides a host cell transformed with the vector.

Transformation with the vector can be performed using transformation techniques known to those skilled in the art. Preferably, methods such as microprojectile bombardment, electroporation, calcium phosphate (CaPO₄) precipitation, calcium chloride (CaCl₂)) precipitation, PEG-mediated fusion, microinjection, and liposome-mediated methods can be used.

The term ‘host cell’ can be used interchangeably with ‘transformant’ and refers to prokaryotic or eukaryotic cells that contain heterologous DNA introduced by any means (e.g., electroporation, calcium phosphate precipitation, microinjection, transformation, viral infection, etc.).

In the present invention, the host cell can be any type of unicellular organism commonly used in the field of cloning, such as prokaryotic microorganisms like various bacteria (e.g., Clostridia, E. coli, etc.), lower eukaryotic microorganisms like yeast, and cells derived from higher eukaryotic organisms including insect cells, plant cells, and mammalian cells, without limitation. Depending on the host cell, the expression level and modification of the protein may vary, so those skilled in the art can select and use the most suitable host cell for their intended purpose.

The present invention also provides a pharmaceutical composition comprising the Bacillus subtilis flagellin variant as an active ingredient.

According to one embodiment of the present invention, the flagellin variant and the fusion protein can exhibit the ability to activate the TLR5 pathway.

Therefore, the fusion protein of the present invention can exhibit preventive, ameliorative, or therapeutic effects on diseases, syndromes, etc., known to be preventable, ameliorable, or treatable through the activation of the TLR5 pathway.

Diseases and syndromes known to be preventable, ameliorable, or treatable through the activation of the TLR5 pathway include damage caused by radiation exposure; reperfusion injury; inflammatory bowel disease; autoimmune disease; viral infection; aging; immune function decline; or cancer.

Therefore, the pharmaceutical composition of the present invention can be characterized as a pharmaceutical composition for preventing or treating damage caused by radiation exposure; preventing or treating reperfusion injury; preventing or treating inflammatory bowel disease; preventing or treating autoimmune disease; preventing or treating viral infection; preventing or treating metabolic disease; preventing or treating aging; enhancing immune function; preventing or treating cancer; preventing or treating liver disease; or preventing or treating colitis.

In particular, the flagellin variant of the present invention can be understood to exhibit preventive, ameliorative, or therapeutic effects on diseases that will be discovered in the future to be preventable, ameliorable, or treatable through the activation of the TLR5 pathway, so the range of diseases targeted by the pharmaceutical composition of the present invention is not particularly limited.

The relationship between the activation of the TLR5 pathway and the treatment of damage caused by radiation exposure, the relationship between the activation of the TLR5 pathway and the treatment of tissue damage caused by reperfusion, the relationship between the activation of the TLR5 pathway and the treatment of inflammatory bowel disease, the relationship between the activation of the TLR5 pathway and the treatment of autoimmune disease, the relationship between the activation of the TLR5 pathway and the treatment of viral infection, the relationship between the activation of the TLR5 pathway and diseases caused by aging, the relationship between the activation of the TLR5 pathway and immune enhancement, and the relationship between the activation of the TLR5 pathway and cancer treatment can be referenced from published literature. In the present invention, the damage caused by radiation exposure can be gastrointestinal syndrome or hematopoietic syndrome caused by radiation exposure.

In the present invention, the diseases caused by aging can be hair loss, cataracts, hernia, colitis, osteoporosis, or osteomalacia caused by aging.

In the present invention, the cancer can be breast cancer, lung cancer, colon cancer, kidney cancer, liver cancer, ovarian cancer, prostate cancer, testicular cancer, urogenital cancer, lymphatic cancer, rectal cancer, pancreatic cancer, esophageal cancer, stomach cancer, cervical cancer, thyroid cancer, skin cancer, leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, histiocytic lymphoma, Burkitt's lymphoma, acute and chronic myeloid leukemia, myelodysplastic syndrome, myeloid leukemia, promyelocytic leukemia, astrocytoma, neuroblastoma, glioma, schwannoma, fibrosarcoma, rhabdomyosarcoma, osteosarcoma, xeroderma pigmentosum, keratoacanthoma, seminoma, follicular thyroid cancer, teratocarcinoma, or gastrointestinal cancer.

In the present invention, the liver disease can be selected from the group consisting of non-alcoholic fatty liver, non-alcoholic steatohepatitis, alcoholic fatty liver, hepatitis, liver fibrosis, liver cirrhosis, liver failure, and liver cancer.

In the present invention, the metabolic disease can be selected from the group consisting of diabetes, metabolic syndrome, insulin resistance, hyperlipidemia, and hypertension.

In the present invention, the colitis can be inflammatory bowel disease or irritable bowel syndrome, and the inflammatory bowel disease can be ulcerative colitis or Crohn's disease.

The pharmaceutical composition of the present invention can be formulated in various ways according to the route of administration in a manner known to those skilled in the art, along with pharmaceutically acceptable carriers in addition to the flagellin variant. “Pharmaceutically acceptable” refers to non-toxic substances that are physiologically acceptable and do not inhibit the action of the active ingredient when administered to humans, and do not cause allergic reactions such as gastrointestinal disorders or dizziness or similar responses. The carrier can include all types of solvents, dispersion media, oil-in-water or water-in-oil emulsions, aqueous compositions, liposomes, microbeads, and microemulsions.

The route of administration can be oral or parenteral. Parenteral administration methods can include, but are not limited to, intravenous, intramuscular, intra-arterial, intramedullary, intradural, intracardiac, transdermal, subcutaneous, intraperitoneal, intranasal, enteral, topical, sublingual, or rectal administration.

When the pharmaceutical composition of the present invention is administered orally, it can be formulated in the form of powders, granules, tablets, pills, sugar-coated tablets, capsules, liquids, gels, syrups, suspensions, wafers, etc., along with suitable carriers for oral administration in a manner known to those skilled in the art. Examples of suitable carriers include sugars such as lactose, dextrose, sucrose, sorbitol, mannitol, xylitol, erythritol, and maltitol; starches such as corn starch, wheat starch, rice starch, and potato starch; celluloses such as cellulose, methylcellulose, sodium carboxymethylcellulose, and hydroxypropylmethylcellulose; and fillers such as gelatin and polyvinylpyrrolidone. Additionally, disintegrants such as cross-linked polyvinylpyrrolidone, agar, alginic acid, or sodium alginate can be added as needed. Furthermore, the pharmaceutical composition can additionally include anti-coagulants, lubricants, wetting agents, flavoring agents, emulsifiers, and preservatives.

Additionally, when administered parenterally, the pharmaceutical composition of the present invention can be formulated in the form of injections, transdermal administration, and nasal inhalation, along with suitable parenteral carriers according to methods known in the art. In the case of injections, they must be sterilized and protected from contamination by microorganisms such as bacteria and fungi. Examples of suitable carriers for injections include, but are not limited to, solvents or dispersion media comprising water, ethanol, polyols (e.g., glycerol, propylene glycol, and liquid polyethylene glycol), mixtures thereof, and/or vegetable oils. More preferably, suitable carriers include isotonic solutions such as Hank's solution, Ringer's solution, phosphate-buffered saline (PBS) containing triethanolamine, or sterile water for injection, 10% ethanol, 40% propylene glycol, and 5% dextrose. To protect the injections from microbial contamination, various antibacterial and antifungal agents such as parabens, chlorobutanol, phenol, sorbic acid, and thimerosal can be additionally included. Furthermore, the injections may additionally include isotonic agents such as sugars or sodium chloride in most cases.

For transdermal administration, forms such as ointments, creams, lotions, gels, external solutions, pastes, liniments, and aerosols are included. “Transdermal administration” means that the pharmaceutical composition is topically applied to the skin, delivering an effective amount of the active ingredient contained in the pharmaceutical composition into the skin. For example, the pharmaceutical composition of the present invention can be administered by formulating it into an injectable form and lightly pricking the skin with a fine 30-gauge needle or directly applying it to the skin. These formulations are described in the literature generally known in pharmaceutical chemistry, such as Remington's Pharmaceutical Science, 15th Edition, 1975, Mack Publishing Company, Easton, Pennsylvania.

For inhalation administration, the flagellin variant can be conveniently delivered in the form of an aerosol spray from a pressurized pack or nebulizer using, for example, dichlorofluoromethane, trichlorofluoromethane, dichlorotetrafluoroethane, carbon dioxide, or other suitable gases. In the case of pressurized aerosols, the dosage unit can be determined by providing a valve that delivers a metered amount. For example, gelatin capsules and cartridges used in inhalers or insufflators can be formulated to contain a powder mixture of the compound and a suitable powder base such as lactose or starch.

Other pharmaceutically acceptable carriers can be referenced from known or published literature. Additionally, the pharmaceutical composition of the present invention can further comprise one or more buffers (e.g., saline or PBS), carbohydrates (e.g., glucose, mannose, sucrose, or dextran), antioxidants, bacteriostats, chelating agents (e.g., EDTA or glutathione), adjuvants (e.g., aluminum hydroxide), suspending agents, thickeners, and/or preservatives.

Furthermore, the pharmaceutical composition of the present invention can be formulated using methods known in the art to provide rapid, sustained, or delayed release of the active ingredient after administration to a mammal.

Additionally, the pharmaceutical composition of the present invention can be administered in combination with known substances effective in preventing or treating each of the listed diseases. The present invention also provides a vaccine adjuvant comprising the Bacillus subtilis flagellin variant as an active ingredient.

One of the most important requirements of a vaccine adjuvant is to have immunoregulatory functions such as regulating the expression of co-stimulatory molecules on the surface of antigen-presenting cells and controlling cytokine secretion induced by antigen-specific T cells. However, PRRs such as TLR5 are distributed on the cell surface or within the cytoplasm of host cells and induce an ‘innate immune response’ and further regulate an ‘adaptive immune response’ through stimulation by various PAMPs. Therefore, TLR5 agonists can be suitable targets for the development of various ‘immunomodulators’, particularly ‘vaccine adjuvants’.

Thus, the fusion protein of the present invention, which has the ability to activate the TLR5 pathway, can significantly enhance the host's immune response to the co-administered antigen by stimulating the TLR5 pathway and augmenting both innate and adaptive immune responses.

The vaccine adjuvant of the present invention can be manufactured by conventional methods well known in the art and may optionally include various additives that can be used in vaccine manufacturing.

The present invention also provides a use of the Bacillus subtilis flagellin variant according to any one of claims 1 to 13 for manufacturing a pharmaceutical composition for treating damage caused by radiation exposure; for treating reperfusion injury; for treating inflammatory bowel disease; for treating autoimmune disease; for treating viral infection; for treating metabolic disease; for treating aging; for enhancing immune function; for treating cancer; for treating liver disease; or for treating colitis.

The present invention also provides a method for treating damage caused by radiation exposure; treating reperfusion injury; treating inflammatory bowel disease; treating autoimmune disease; treating viral infection; treating metabolic disease; treating aging; enhancing immune function; treating cancer; treating liver disease; or treating colitis, comprising administering an effective amount of a pharmaceutical composition comprising the Bacillus subtilis flagellin variant according to any one of claims 1 to 13 as an active ingredient to a subject in need thereof.

The ‘effective amount’ of the present invention refers to an amount that, when administered to a subject, exhibits an effect of improving, treating, detecting, diagnosing, or inhibiting or reducing cancer or the diseases, and the ‘subject’ can be an animal, preferably a mammal, particularly an animal including a human, and may also be cells, tissues, organs, etc., derived from animals. The subject may be a patient in need of the effect.

The ‘treatment’ of the present invention comprehensively refers to improving symptoms caused by cancer or the diseases, which may include curing, substantially preventing, or improving the condition, and may include alleviating, curing, or preventing one or more symptoms or most symptoms caused by the disease, but is not limited thereto.

In this specification, the term “comprising” is used in the same sense as “including” or “characterized by” and does not exclude additional components or steps not specifically mentioned in the composition or method according to the present invention. The term “consisting of” means excluding additional elements, steps, or components not specifically described. The term “consisting essentially of” means that the composition or method may include substances or steps that do not substantially affect the basic characteristics of the described material or step.

### Effect of the Invention

The Bacillus subtilis flagellin variant provided by the present invention not only has reduced immunogenicity in vivo compared to the wild-type, but also has improved storage stability, making it highly useful for the development of therapeutics and/or vaccine adjuvants for diseases that can be prevented, ameliorated, or treated through TLR5 activation.

### BRIEF DESCRIPTION OF THE DRAWINGS

FIGS. 1a to 1c are diagrams showing schematic structures of wild-type Bsflagellin and Bsflagellin variant proteins prepared in the examples of the present invention.

FIG. 2 is a result of evaluating the signal intensity of NF-kB after administering the Bsflagellin variant BM-03 and wild-type Bsflagellin to NF-kB reporter mice.

### DETAILED DESCRIPTION OF THE INVENTION

Hereinafter, the present invention will be described in detail with reference to the following examples. However, the following examples are merely illustrative of the present invention and are not intended to limit the present invention.

### Experimental Methods

### 1. Immunogenicity Prediction and Variant Design Using a Computer

(1) Based on the sequence information of the target protein, regions that can elicit an immune response by B-cells were predicted.
(2) Typically, the sequence information was put into the BepiPred 2.0 program, and regions with a cut-off value of 0.6 or higher were analyzed as immunogenic regions.
(3) After mathematically calculating the area of regions exceeding the cut-off, the relative immunogenicity was predicted based on the immunogenicity value of wild-type Bacillus flagellin. Accordingly, variants comprising the mutations shown in Table 1 below were designed from the wild-type Bsflagellin of SEQ ID NO: 1:

TABLE 1

| Name | Mutation location(based on SEQ ID NO: 1) |
|---|---|
| Bsflagellin | WT (SEQ ID NO: 1) |
| BM01 | deletion: M1~S31 |
| BM02 | deletion: T100, D144, G145, F146, A181, A182, T183 |
| BM03 | deletion: M1~S31, T100, D144, G145, F146, A181, A182, T183 |
| BM04 | deletion: M1~S31, T100, D144, G145, F146, A181, A182, T183, D179, F180 |
| BM05 | deletion: M1~G33, T100, D144, G145, F146, A181, A182, T183 |
| BM06 | deletion: M1~D41, T100, D144, G145, F146, A181, A182, T183 |
| BM07 | deletion: M1~43, T100, D144, G145, F146, A181, A182, T183 |
| BM08 | deletion: M1~E50, T100, D144, G145, F146, A181, A182, T183 |
| BM09 | deletion: M1~S31, S164~D179, T100, D144, G145, F146, A181, A182, T183 |
| BM10 | deletion: M1~S31, A152~D186, T100, D144, G145, F146, [symbol] |
| BM11 | deletion: M1~S31, T100, D144, G145, F146, A181, A182, T183, V270~R275 |
| BM12 | deletion: M1~S31, T100, D144, G145, F146, A181, A182, T183, Q266~R275 |
| BM13 | deletion: M1~S31, T100, D144, G145, F146, A181, A182, T183, D236~R275 |
| BM14 | deletion: M1~S31, T100, D144, G145, F146, A181, A182, T183, A230~R275 |
| BM15 | deletion: M1~E50, T100, D144, G145, F146, A181, A182, T183, D236~R275 |
| BM16 | deletion: M1~S31, T100, D144, G145, F146, A181, A182, T183 M243~S253 |
| BM17 | deletion: M1~S31, T100, D144, G145, F146, A181, A182, T183, M239~S253 |
| BM18 | deletion: M1~S31, T100, D144, G145, F146, A181, A182, T183, M243~L260 |
| BM19 | deletion: M1~S31, T100, D144, G145, F146, A181, A182, T183, D236~S253 |
| BS01 | Deletion domain of BM03 + L34C, D236C |
| BS02 | Deletion domain of BM03 + S49C, E231C |
| BS03 | Deletion domain of BM03 + M52C, E231C |
| BS04 | Deletion domain of BM03 + L59C, G224C |
| BD01 | Deletion domain of BM03 + Q103A, Q104A |
| BD02 | Deletion domain of BM03 + G151A |
| BD03 | Deletion domain of BM03 + G155A |
| BD04 | Deletion domain of BM03 + D176A |
| BD05 | Deletion domain of BM03 + D238A |
| BD06 | Deletion domain of BM03 + Q103A, Q104A, G155A |
| BD07 | Deletion domain of BM03 + Q103A, Q104A, D176A |
| BD08 | Deletion domain of BM03 + Q103A, Q104A, D238A |
| BD09 | Deletion domain of BM03 + Q103A, Q104A, G151A, D176A |
| BD10 | Deletion domain of BM03 + Q103A, Q104A, G151A, M239A |
| BA01 | A85K |
| BA02 | A85N |
| BA03 | I86N |
| BA04 | L118R |
| BE01 | R275Q |
| BC01 | Deletion domain of BM03 + A85N |
| BC02 | Deletion domain of BM03 + R275Q |
| BC03 | Deletion domain of BM03 + A85N, R275Q |
| BC04 | Deletion domain of BM04 + A85N |
| BC05 | Deletion domain of BM04 + R275Q |
| BC06 | Deletion domain of BM04 + A85N, R275Q |

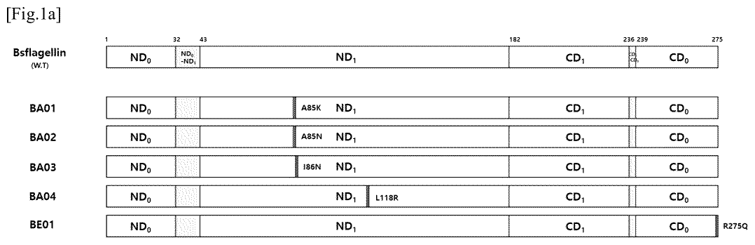

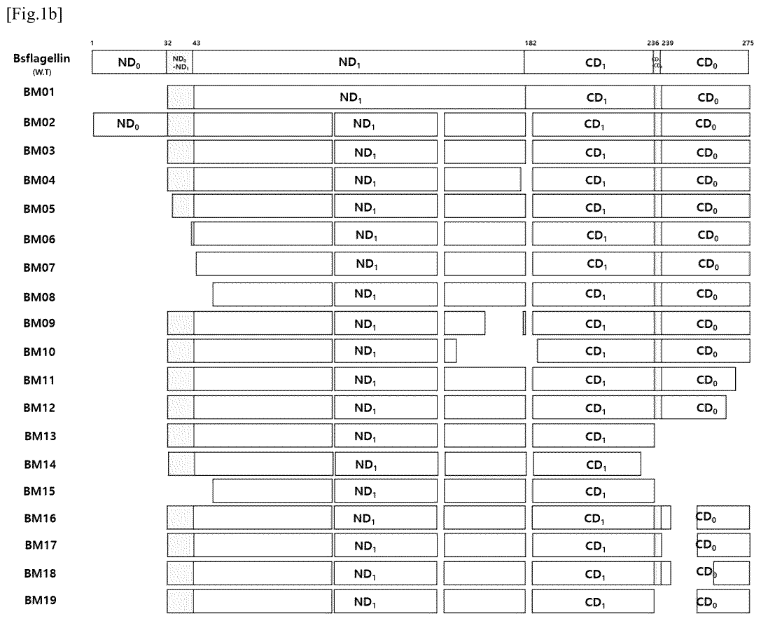

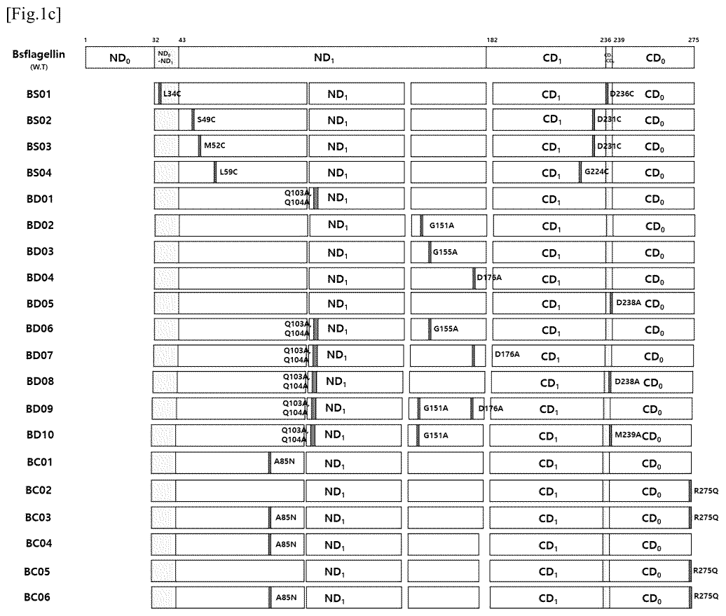

The schematic diagram of the protein structure of each Bsflagellin variant and the wild-type Bsflagellin is shown in FIG. 1.

### 2. Gene Cloning

Plasmids comprising the sequences described in Table 2 were produced, and each protein was produced using these plasmids. Cloning was performed in the following order.

### PCR

(1) Site-directed mutagenesis by PCR was performed using primers matching the ORF produced by Macrogen and using wild-type Bacillus flagellin as the template DNA.
(2) PCR was performed by setting the PCR conditions according to the primer sequence and the sequence to be synthesized.

After performing PCR product preparation, 2 μl of 6X Dyne loading star dye was added to the PCR fragment DNA, and the size and presence of bands of the PCR product were confirmed by gel electrophoresis.

### Restriction

(1) 1 μl of TaKaRa Dpn1 (1235A) was added to the PCR product and incubated at 37° C. for 30 minutes. After 30 minutes, PCR prep was performed.

### Ligation

(1) A total of 10 μl solution was prepared by combining the mixture of the obtained DNA and vector, and Soll of Takara DNA Ligase Kit Ver. 2.1 (6022) at a ratio of 1:1, and incubated at 25° C. for 30 minutes.

### Transformation

(1) 50 μl of DH5a (RH617) and BL21 (DE3) (CP110) competent cells were thawed for 15 minutes, and the ligated plasmids were added and incubated on ice for 20 minutes.
(2) Heat shock was given for 50 seconds in a 42° C. heating block, followed by incubation on ice for 5 minutes.
(3) 50 μl of LB was added to the cells of step (2), and the cells were grown by incubating at 37° C. for 30 minutes.
(4) The cells from step (3) were added and spread on a kanamycin (+) LB Agar plate and incubated at 37° C. for 18 hours.
(5) A single colony from the plate in step (4) was selected and grown for 16 hours, followed by 5 plasmid prep.
(6) The plasmid obtained using the prep was sequenced (Macrogen) and confirmed the completion of cloning.

The sequences of proteins and DNA produced using the primers in Table 2 are shown in Table 2.

TABLE 2

| Name | Classification | Sequence |
|---|---|---|
| BM01 | Amino acid | SGLRINRAGDDAAGLAISEKMRGQIRGLEMASKNSQDGISLIQ |
| | sequence | TAEGALTETHAILQRMRELTVQAGNTGTQQAEDLGAIKDEMDA |
| | (SEQ ID | LIEEIDGISNRTEFNGKKLLDGTNSTDGFTFQIGANAGQQLNV |
| | NO: 2) | KIDSMSSTALGVNALDVTDFAATAFDDQLKSIDTAINTVSTQR |
| | | AKLGAVQNRLEHTINNLGASGENLTAAESRIRDVDMAKEMSEF |
| | | TKNNILSQASQAMLAQANQQPQNVLQLLR |
| BM02 | Amino acid | MRINHNIAALNTLNRLGSNNGAAQKNMEKLSSGLRINRAGDDA |
| | sequence | AGLAISEKMRGQIRGLEMASKNSQDGISLIQTAEGALTETHAI |
| | (SEQ ID | LQRMRELTVQAGNGTQQAEDLGAIKDEMDALIEEIDGISNRTE |
| | NO: 3) | FNGKKLLDGTNSTTFQIGANAGQQLNVKIDSMSSTALGVNALD |
| | | VTDFAFDDQLKSIDTAINTVSTQRAKLGAVQNRLEHTINNLGA |
| | | SGENLTAAESRIRDVDMAKEMSEFTKNNILSQASQAMLAQANQ |
| | | QPQNVLQLLR |
| BM03 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 4) | MSSTALGVNALDVTDFAFDDQLKSIDTAINTVSTQRAKLGAVQ |
| | | NRLEHTINNLGASGENLTAAESRIRDVDMAKEMSEFTKNNILS |
| | | QASQAMLAQANQQPQNVLQLLR |
| BM04 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 5) | MSSTALGVNALDVTAFDDQLKSIDTAINTVSTQRAKLGAVQNR |
| | | LEHTINNLGASGENLTAAESRIRDVDMAKEMSEFTKNNILSQA |
| | | SQAMLAQANQQPQNVLQLLR |
| BM05 | Amino acid | LRINRAGDDAAGLAISEKMRGQIRGLEMASKNSQDGISLIQTA |
| | sequence | EGALTETHAILQRMRELTVQAGNGTQQAEDLGAIKDEMDALIE |
| | (SEQ ID | EIDGISNRTEFNGKKLLDGTNSTTFQIGANAGQQLNVKIDSMS |
| | NO: 6) | STALGVNALDVTDFAFDDQLKSIDTAINTVSTQRAKLGAVQNR |
| | | LEHTINNLGASGENLTAAESRIRDVDMAKEMSEFTKNNILSQA |
| | | SQAMLAQANQQPQNVLQLLR |
| BM06 | Amino acid | DAAGLAISEKMRGQIRGLEMASKNSQDGISLIQTAEGALTETH |
| | sequence | AILQRMRELTVQAGNGTQQAEDLGAIKDEMDALIEEIDGISNR |
| | (SEQ ID | TEFNGKKLLDGTNSTTFQIGANAGQQLNVKIDSMSSTALGVNA |
| | NO: 7) | LDVTDFAFDDQLKSIDTAINTVSTQRAKLGAVQNRLEHTINNL |
| | | GASGENLTAAESRIRDVDMAKEMSEFTKNNILSQASQAMLAQA |
| | | NQQPQNVLQLLR |
| BM07 | Amino acid | AGLAISEKMRGQIRGLEMASKNSQDGISLIQTAEGALTETHAI |
| | sequence | LQRMRELTVQAGNGTQQAEDLGAIKDEMDALIEEIDGISNRTE |
| | (SEQ ID | FNGKKLLDGTNSTTFQIGANAGQQLNVKIDSMSSTALGVNALD |
| | NO: 8) | VTDFAFDDQLKSIDTAINTVSTQRAKLGAVQNRLEHTINNLGA |
| | | SGENLTAAESRIRDVDMAKEMSEFTKNNILSQASQAMLAQANQ |
| | | QPQNVLQLLR |
| BM08 | Amino acid | KMRGQIRGLEMASKNSQDGISLIQTAEGALTETHAILQRMREL |
| | sequence | TVQAGNGTQQAEDLGAIKDEMDALIEEIDGISNRTEFNGKKLL |
| | (SEQ ID | DGTNSTTFQIGANAGQQLNVKIDSMSSTALGVNALDVTDFAFD |
| | NO: 9) | DQLKSIDTAINTVSTQRAKLGAVQNRLEHTINNLGASGENLTA |
| | | AESRIRDVDMAKEMSEFTKNNILSQASQAMLAQANQQPQNVLQ |
| | | LLR |
| BM09 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDF |
| | NO: 10) | AFDDQLKSIDTAINTVSTQRAKLGAVQNRLEHTINNLGASGEN |
| | | LTAAESRIRDVDMAKEMSEFTKNNILSQASQAMLAQANQQPQN |
| | | VLQLLR |
| BM10 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGDQLKSIDTAINTV |
| | NO: 11) | STQRAKLGAVQNRLEHTINNLGASGENLTAAESRIRDVDMAKE |
| | | MSEFTKNNILSQASQAMLAQANQQPQNVLQLLR |
| BM11 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 12) | MSSTALGVNALDVTDFAFDDQLKSIDTAINTVSTQRAKLGAVQ |
| | | NRLEHTINNLGASGENLTAAESRIRDVDMAKEMSEFTKNNILS |
| | | QASQAMLAQANQQPQN |
| BM12 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 13) | MSSTALGVNALDVTDFAFDDQLKSIDTAINTVSTQRAKLGAVQ |
| | | NRLEHTINNLGASGENLTAAESRIRDVDMAKEMSEFTKNNILS |
| | | QASQAMLAQANQ |
| BM13 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 14) | MSSTALGVNALDVTDFAFDDQLKSIDTAINTVSTQRAKLGAVQ |
| | | NRLEHTINNLGASGENLTAAESRIR |
| BM14 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 15) | MSSTALGVNALDVTDFAFDDQLKSIDTAINTVSTQRAKLGAVQ |
| | | NRLEHTINNLGASGENLTA |
| BM15 | Amino acid | KMRGQIRGLEMASKNSQDGISLIQTAEGALTETHAILQRMREL |
| | sequence | TVQAGNGTQQAEDLGAIKDEMDALIEEIDGISNRTEFNGKKLL |
| | (SEQ ID | DGTNSTTFQIGANAGQQLNVKIDSMSSTALGVNALDVTDFAFD |
| | NO: 16) | DQLKSIDTAINTVSTQRAKLGAVQNRLEHTINNLGASGENLTA |
| | | AESRIR |
| BM16 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 17) | MSSTALGVNALDVTDFAFDDQLKSIDTAINTVSTQRAKLGAVQ |
| | | NRLEHTINNLGASGENLTAAESRIRDVDMAKEQASQAMLAQAN |
| | | QQPQNVLQLLR |
| BM17 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 18) | MSSTALGVNALDVTDFAFDDQLKSIDTAINTVSTQRAKLGAVQ |
| | | NRLEHTINNLGASGENLTAAESRIRDVDQASQAMLAQANQQPQ |
| | | NVLQLLR |
| BM18 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 19) | MSSTALGVNALDVTDFAFDDQLKSIDTAINTVSTQRAKLGAVQ |
| | | NRLEHTINNLGASGENLTAAESRIRDVDMAKEAQANQQPQNVL |
| | | QLLR |
| BM19 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 20) | MSSTALGVNALDVTDFAFDDQLKSIDTAINTVSTQRAKLGAVQ |
| | | NRLEHTINNLGASGENLTAAESRIRQASQAMLAQANQQPQNVL |
| | | QLLR |
| BS01 | Amino acid | SGCRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 21) | MSSTALGVNALDVTDFAFDDQLKSIDTAINTVSTQRAKLGAVQ |
| | | NRLEHTINNLGASGENLTAAESRIRCVDMAKEMSEFTKNNILS |
| | | QASQAMLAQANQQPQNVLQLLR |
| BS02 | Amino acid | SGLRINRAGDDAAGLAICEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 22) | MSSTALGVNALDVTDFAFDDQLKSIDTAINTVSTQRAKLGAVQ |
| | | NRLEHTINNLGASGENLTAACSRIRDVDMAKEMSEFTKNNILS |
| | | QASQAMLAQANQQPQNVLQLLR |
| BS03 | Amino acid | SGLRINRAGDDAAGLAISEKCRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 23) | MSSTALGVNALDVTDFAFDDQLKSIDTAINTVSTQRAKLGAVQ |
| | | NRLEHTINNLGASGENLTAACSRIRDVDMAKEMSEFTKNNILS |
| | | QASQAMLAQANQQPQNVLQLLR |
| BS04 | Amino acid | SGLRINRAGDDAAGLAISEKMRGQIRGCEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 24) | MSSTALGVNALDVTDFAFDDQLKSIDTAINTVSTQRAKLGAVQ |
| | | NRLEHTINNLGASCENLTAAESRIRDVDMAKEMSEFTKNNILS |
| | | QASQAMLAQANQQPQNVLQLLR |
| BD01 | Amino acid | SGLRINRAGDDAAGLAISEKMRGQIRGLEMASKNSQDGISLIQ |
| | sequence | TAEGALTETHAILQRMRELTVQAGNGTAAAEDLGAIKDEMDAL |
| | (SEQ ID | IEEIDGISNRTEFNGKKLLDGTNSTTFQIGANAGQQLNVKIDS |
| | NO: 25) | MSSTALGVNALDVTDFAFDDQLKSIDTAINTVSTQRAKLGAVQ |
| | | NRLEHTINNLGASGENLTAAESRIRDVDMAKEMSEFTKNNILS |
| | | QASQAMLAQANQQPQNVLQLLR |
| BD02 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIAANAGQQLNVKIDS |
| | NO: 26) | MSSTALGVNALDVTDFAFDDQLKSIDTAINTVSTQRAKLGAVQ |
| | | NRLEHTINNLGASGENLTAAESRIRDVDMAKEMSEFTKNNILS |
| | | QASQAMLAQANQQPQNVLQLLR |
| BD03 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAAQQLNVKIDS |
| | NO: 27) | MSSTALGVNALDVTDFAFDDQLKSIDTAINTVSTQRAKLGAVQ |
| | | NRLEHTINNLGASGENLTAAESRIRDVDMAKEMSEFTKNNILS |
| | | QASQAMLAQANQQPQNVLQLLR |
| BD04 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 28) | MSSTALGVNALAVTDFAFDDQLKSIDTAINTVSTQRAKLGAVQ |
| | | NRLEHTINNLGASGENLTAAESRIRDVDMAKEMSEFTKNNILS |
| | | QASQAMLAQANQQPQNVLQLLR |
| BD05 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 29) | MSSTALGVNALDVTDFAFDDQLKSIDTAINTVSTQRAKLGAVQ |
| | | NRLEHTINNLGASGENLTAAESRIRDVAMAKEMSEFTKNNILS |
| | | QASQAMLAQANQQPQNVLQLLR |
| BD06 | Amino acid | SGLRINRAGDDAAGLAISEKMRGQIRGLEMASKNSQDGISLIQ |
| | sequence | TAEGALTETHAILQRMRELTVQAGNGTAAAEDLGAIKDEMDAL |
| | (SEQ ID | IEEIDGISNRTEFNGKKLLDGTNSTTFQIGANAAQQLNVKIDS |
| | NO: 30) | MSSTALGVNALDVTDFAFDDQLKSIDTAINTVSTQRAKLGAVQ |
| | | NRLEHTINNLGASGENLTAAESRIRDVDMAKEMSEFTKNNILS |
| | | QASQAMLAQANQQPQNVLQLLR |
| BD07 | Amino acid | SGLRINRAGDDAAGLAISEKMRGQIRGLEMASKNSQDGISLIQ |
| | sequence | TAEGALTETHAILQRMRELTVQAGNGTAAAEDLGAIKDEMDAL |
| | (SEQ ID | IEEIDGISNRTEFNGKKLLDGTNSTTFQIGANAGQQLNVKIDS |
| | NO: 31) | MSSTALGVNALAVTDFAFDDQLKSIDTAINTVSTQRAKLGAVQ |
| | | NRLEHTINNLGASGENLTAAESRIRDVDMAKEMSEFTKNNILS |
| | | QASQAMLAQANQQPQNVLQLLR |
| BD08 | Amino acid | SGLRINRAGDDAAGLAISEKMRGQIRGLEMASKNSQDGISLIQ |
| | sequence | TAEGALTETHAILQRMRELTVQAGNGTAAAEDLGAIKDEMDAL |
| | (SEQ ID | IEEIDGISNRTEFNGKKLLDGTNSTTFQIGANAGQQLNVKIDS |
| | NO: 32) | MSSTALGVNALDVTDFAFDDQLKSIDTAINTVSTQRAKLGAVQ |
| | | NRLEHTINNLGASGENLTAAESRIRDVAMAKEMSEFTKNNILS |
| | | QASQAMLAQANQQPQNVLQLLR |
| BD09 | Amino acid | SGLRINRAGDDAAGLAISEKMRGQIRGLEMASKNSQDGISLIQ |
| | sequence | TAEGALTETHAILQRMRELTVQAGNGTAAAEDLGAIKDEMDAL |
| | (SEQ ID | IEEIDGISNRTEFNGKKLLDGTNSTTFQIAANAGQQLNVKIDS |
| | NO: 33) | MSSTALGVNALAVTDFAFDDQLKSIDTAINTVSTQRAKLGAVQ |
| | | NRLEHTINNLGASGENLTAAESRIRDVDMAKEMSEFTKNNILS |
| | | QASQAMLAQANQQPQNVLQLLR |
| BD10 | Amino acid | SGLRINRAGDDAAGLAISEKMRGQIRGLEMASKNSQDGISLIQ |
| | sequence | TAEGALTETHAILQRMRELTVQAGNGTAAAEDLGAIKDEMDAL |
| | (SEQ ID | IEEIDGISNRTEFNGKKLLDGTNSTTFQIAANAGQQLNVKIDS |
| | NO: 34) | MSSTALGVNALDVTDFAFDDQLKSIDTAINTVSTQRAKLGAVQ |
| | | NRLEHTINNLGASGENLTAAESRIRDVDAAKEMSEFTKNNILS |
| | | QASQAMLAQANQQPQNVLQLLR |
| BA01 | Amino acid | MRINHNIAALNTLNRLGSNNGAAQKNMEKLSSGLRINRAGDDA |
| | sequence | AGLAISEKMRGQIRGLEMASKNSQDGISLIQTAEGALTETHKI |
| | (SEQ ID | LQRMRELTVQAGNTGTQQAEDLGAIKDEMDALIEEIDGISNRT |
| | NO: 35) | EFNGKKLLDGTNSTDGFTFQIGANAGQQLNVKIDSMSSTALGV |
| | | NALDVTDFAATAFDDQLKSIDTAINTVSTQRAKLGAVQNRLEH |
| | | TINNLGASGENLTAAESRIRDVDMAKEMSEFTKNNILSQASQA |
| | | MLAQANQQPQNVLQLLR |
| BA02 | Amino acid | MRINHNIAALNTLNRLGSNNGAAQKNMEKLSSGLRINRAGDDA |
| | sequence | AGLAISEKMRGQIRGLEMASKNSQDGISLIQTAEGALTETHNI |
| | (SEQ ID | LQRMRELTVQAGNTGTQQAEDLGAIKDEMDALIEEIDGISNRT |
| | NO: 36) | EFNGKKLLDGTNSTDGFTFQIGANAGQQLNVKIDSMSSTALGV |
| | | NALDVTDFAATAFDDQLKSIDTAINTVSTQRAKLGAVQNRLEH |
| | | TINNLGASGENLTAAESRIRDVDMAKEMSEFTKNNILSQASQA |
| | | MLAQANQQPQNVLQLLR |
| BA03 | Amino acid | MRINHNIAALNTLNRLGSNNGAAQKNMEKLSSGLRINRAGDDA |
| | sequence | AGLAISEKMRGQIRGLEMASKNSQDGISLIQTAEGALTETHAN |
| | (SEQ ID | LQRMRELTVQAGNTGTQQAEDLGAIKDEMDALIEEIDGISNRT |
| | NO: 37) | EFNGKKLLDGTNSTDGFTFQIGANAGQQLNVKIDSMSSTALGV |
| | | NALDVTDFAATAFDDQLKSIDTAINTVSTQRAKLGAVQNRLEH |
| | | TINNLGASGENLTAAESRIRDVDMAKEMSEFTKNNILSQASQA |
| | | MLAQANQQPQNVLQLLR |
| BA04 | Amino acid | MRINHNIAALNTLNRLGSNNGAAQKNMEKLSSGLRINRAGDDA |
| | sequence | AGLAISEKMRGQIRGLEMASKNSQDGISLIQTAEGALTETHAI |
| | (SEQ ID | LQRMRELTVQAGNTGTQQAEDLGAIKDEMDARIEEIDGISNRT |
| | NO: 38) | EFNGKKLLDGTNSTDGFTFQIGANAGQQLNVKIDSMSSTALGV |
| | | NALDVTDFAATAFDDQLKSIDTAINTVSTQRAKLGAVQNRLEH |
| | | TINNLGASGENLTAAESRIRDVDMAKEMSEFTKNNILSQASQA |
| | | MLAQANQQPQNVLQLLR |
| BE01 | Amino acid | MRINHNIAALNTLNRLGSNNGAAQKNMEKLSSGLRINRAGDDA |
| | sequence | AGLAISEKMRGQIRGLEMASKNSQDGISLIQTAEGALTETHAI |
| | (SEQ ID | LQRMRELTVQAGNTGTQQAEDLGAIKDEMDALIEEIDGISNRT |
| | NO: 39) | EFNGKKLLDGTNSTDGFTFQIGANAGQQLNVKIDSMSSTALGV |
| | | NALDVTDFAATAFDDQLKSIDTAINTVSTQRAKLGAVQNRLEH |
| | | TINNLGASGENLTAAESRIRDVDMAKEMSEFTKNNILSQASQA |
| | | MLAQANQQPQNVLQLLQ |
| BC01 | Amino acid | SGLRINRAGDDAAGLAISEKMRGQIRGLEMASKNSQDGISLIQ |
| | sequence | TAEGALTETHNILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 40) | MSSTALGVNALDVTDFAFDDQLKSIDTAINTVSTQRAKLGAVQ |
| | | NRLEHTINNLGASGENLTAAESRIRDVDMAKEMSEFTKNNILS |
| | | QASQAMLAQANQQPQNVLQLLR |
| BC02 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 41) | MSSTALGVNALDVTDFAFDDQLKSIDTAINTVSTQRAKLGAVQ |
| | | NRLEHTINNLGASGENLTAAESRIRDVDMAKEMSEFTKNNILS |
| | | QASQAMLAQANQQPQNVLQLLQ |
| BC03 | Amino acid | SGLRINRAGDDAAGLAISEKMRGQIRGLEMASKNSQDGISLIQ |
| | sequence | TAEGALTETHNILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 42) | MSSTALGVNALDVTDFAFDDQLKSIDTAINTVSTQRAKLGAVQ |
| | | NRLEHTINNLGASGENLTAAESRIRDVDMAKEMSEFTKNNILS |
| | | QASQAMLAQANQQPQNVLQLLQ |
| BC04 | Amino acid | SGLRINRAGDDAAGLAISEKMRGQIRGLEMASKNSQDGISLIQ |
| | sequence | TAEGALTETHNILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 43) | MSSTALGVNALDVTAFDDQLKSIDTAINTVSTQRAKLGAVQNR |
| | | LEHTINNLGASGENLTAAESRIRDVDMAKEMSEFTKNNILSQA |
| | | SQAMLAQANQQPQNVLQLLR |
| BC05 | Amino acid | SGLRINRAGDDAAGLAISEKMRGQIRGLEMASKNSQDGISLIQ |
| | sequence | TAEGALTETHAILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 44) | MSSTALGVNALDVTAFDDQLKSIDTAINTVSTQRAKLGAVQNR |
| | | LEHTINNLGASGENLTAAESRIRDVDMAKEMSEFTKNNILSQA |
| | | SQAMLAQANQQPQNVLQLLQ |
| BC06 | Amino acid | SGLRINRAGDDAAGLAISEKMRGQIRGLEMASKNSQDGISLIQ |
| | sequence | TAEGALTETHNILQRMRELTVQAGNGTQQAEDLGAIKDEMDAL |
| | (SEQ ID | IEEIDGISNRTEFNGKKLLDGTNSTTFQIGANAGQQLNVKIDS |
| | NO: 45) | MSSTALGVNALDVTAFDDQLKSIDTAINTVSTQRAKLGAVQNR |
| | | LEHTINNLGASGENLTAAESRIRDVDMAKEMSEFTKNNILSQA |
| | | SQAMLAQANQQPQNVLQLLQ |

### 3. Protein Expression

### Culture & Cell Harvest

(1) 10 μl of Bsflagellin variant seed culture was inoculated into 10 ml of LB medium containing 50 μM kanamycin.
(2) The culture was incubated overnight at 170 rpm in a 37° C. shaking incubator.
(3) 9 ml of the culture from step (2) was added to 1.2 L of medium containing 50 μM kanamycin.
(4) The culture was grown at 37° C. until the OD (600 nm) reached 0.5-0.7, and IPTG was added to a final concentration of 0.5 mM.
(5) The temperature was lowered to 17° C., and protein expression was induced for 18 hours.
(6) Upon completion of the culture, pellets were harvested by centrifugation at 8000 rpm for 7 minutes.

### 4. Protein Purification

### Cell Lysis & Sonication

(1) 60 ml of cell suspension buffer (20 mM sodium phosphate, pH 7.6, 150 mM NaCl) was added to 5 g of the pellet obtained after culture, and the cells were resuspended using a pipette aid.
(2) The cells were sonicated at Amp: 52%, Pulser: 2 sec/1 sec, Time: 23 min (on time).
(3) The sonicated sample was centrifuged at 10000 rpm for 30 minutes, and the supernatant was collected.

### IMAC (his Affinity Chromatography)

(1) 5 ml of Ni-NTA agarose resin (Qiagen, 30210) was washed in an Econo-column (Bio-Rad, 7374252), and shaking-incubated with the lysed protein supernatant at room temperature for 2 hours.
(2) Washing with imidazole concentration gradient (50 mM/100 mM/250 mM/500 mM imidazole) and elution (elution buffer; 20 mM sodium phosphate, pH 7.6, 150 mM NaCl, 1 M imidazole) were performed to obtain purified proteins.
(3) Each fraction sample was analyzed by SDS-PAGE to confirm the position and purity of the target protein.
(4) The target protein was exchanged into 20 mM sodium phosphate, pH 7.6, 150 mM NaCl buffer (using a 10K Centricon tube).

### His-Tag Cleavage & LPS Removal

(1) 2 μl of thrombin (BPS bio, 80111) was added to the purified sample, which then were incubated with shaking at 37° C. for 1 hour.
(2) After incubation, 1% (v/v) Triton X-114 was added, mixed well with a vortexer, and incubated on ice for 10 minutes.
(3) After 10 minutes, the sample was transferred to a 37° C. heating block and incubated for 10 minutes.
(4) The sample from step (3) was centrifuged at 15000 rpm, 37° C., for 10 minutes to pellet down Triton X-114 and LPS.
(5) Steps (2) to (4) were repeated twice, and the final sample was diluted in 10 times the volume of 20 mM sodium phosphate, pH 7.6, 10 mM NaCl buffer.

### IEX (Anion Exchange Chromatography)

(1) The Hi-trap™ QHP 5 ml column (17115301) was connected to the Akta PURE 150 equipment after CIP and equilibrated with equalization buffer (20 mM sodium phosphate, pH 7.6, 10 mM NaCl) sufficiently for more than 5 CV.
(2) The protein was loaded using a sample pump, and after equilibration, fractionation was performed with a salt gradient (A; 20 mM sodium phosphate, pH 7.6, 10 mM NaCl, B; 20 mM sodium phosphate, pH 7.6, 1 M NaCl).
(3) Each fraction was analyzed by SDS-PAGE, and only the fractions containing the sample were selected.
(4) Only the fractions containing the target sample were separated, and buffer exchange was performed with 1×PBS pH 7.4 buffer (dialysis tube: SnakeSkin™ Dialysis Tubing, 10K MWCO, Pierce, 68100), 4° C., overnight.
(5) The concentration of the obtained proteins was measured using a nano-drop device, and the total amount of the obtained proteins was measured and recorded per liter of culture medium.

### 6. Protein Productivity Analysis

(1) The amount of proteins obtained per liter of culture medium was recorded according to the variant.
(2) The relative productivity was compared and analyzed based on the amount of protein that can be obtained per liter during the purification of wild-type Bacillus flagellin, and the results were shown in a table.

### 7. Protein Stability Comparison Experiment

(1) 5 μg of protein was aliquoted equally and incubated at 37° C. for 0 week, 1 week, and 2 weeks.
(2) The changes in protein patterns were compared by SDS-PAGE for each incubation period.
(3) The changes in the intensity of the major band were graphed using the Image J program.
(4) The stability was compared by measuring the remaining amount of the main peak after 0 week, 1 week, and 2 weeks compared to the initial main peak area.

### 8. In Vivo Imaging Using NF-kB Reporter Mouse (IVIS)

(1) The substance and PBS were subcutaneously injected into NF-kB reporter mice (B6.129-Gt (ROSA) 26Sortm1 (NF-kBp-Luciferase-dTomato) HLee) at 5 μg/mouse once every 2 weeks for a total of 3 times over 6 weeks.
(2) 3 hours after substance injection, 150 mg/kg of luciferin (D-luciferin salt lot #122799 PerkinElmer) was intraperitoneally injected 10-15 minutes before fluorescence detection, and anesthesia was performed with 2% to 2.5% (O₂) isoflurane (Hana Pharmaceutical).
(3) The anesthetized mouse was imaged using the IVIS™ imaging system (Perkin Elmer) while maintaining anesthesia at 1.5% to 1.8% (O₂), and fluorescence detection images were obtained up to the neck of the mouse.

### Experimental Results

### 1. Immunogenicity Evaluation

The results of the immunogenicity evaluation of each recombinant protein are shown in Table 3 below.

TABLE 3

| | Immunogenicity |
|---|---|
| Name | (% compared to WT Bsflagellin) |
| Bsflagellin(WT) | 100 |
| BM01 | 82.11 |
| BM02 | 95.25 |
| BM03 | 81.75 |
| BM04 | 139.98 |
| BM05 | 69.91 |
| BM06 | 109.24 |
| BM07 | 94.12 |
| BM08 | 34.22 |
| BM09 | 163.24 |
| BM10 | 43.04 |
| BM11 | 25.81 |
| BM12 | 79.38 |
| BM13 | 87.28 |
| BM14 | 113.50 |
| BM15 | 127.81 |
| BM16 | 125.26 |
| BM17 | 60.02 |
| BM18 | 55.97 |
| BM19 | 42.97 |
| BS01 | 86.64 |
| BS02 | 47.22 |
| BS03 | 60.37 |
| BS04 | 60.33 |
| BD01 | 38.61 |
| BD02 | 42.71 |
| BD03 | 29.98 |
| BD04 | 17.10 |
| BA01 | 105.18 |
| BA02 | 123.87 |
| BA03 | 127.83 |
| BA04 | 126.61 |
| BE01 | 65.03 |
| BC01 | 42.76 |
| BC02 | 82.40 |
| BC03 | 25.64 |
| BC04 | 170.62 |
| BC05 | 167.12 |
| BC06 | 211.41 |

The TLR5 agonist activity of each of the produced Bsflagellin variants and Bsflagellin WT was evaluated according to a conventionally reported method, and it was confirmed that the Bsflagellin variants exhibited activity at a level equivalent to that of Bsflagellin WT.

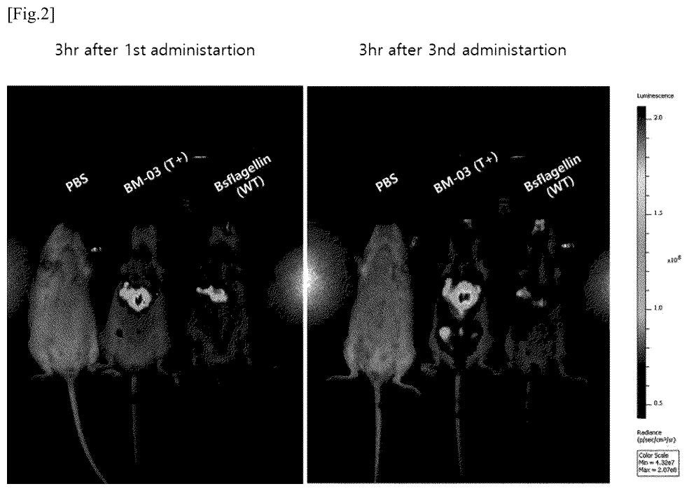

Next, after multiple administrations (third administration) of Bsflagellin WT and the variant BM03, the change in NF-kB signal intensity in NF-kB reporter mice was observed to confirm the TLR5-specific NF-kB activity of BM03 compared to Bsflagellin (WT) along with the generation of neutralizing antibodies. The results are shown in FIG. 2.

As shown in FIG. 2, it was confirmed that the TLR5-specific NF-kB activity of the variant BM03 was maintained at a higher level compared to Bsflagellin WT. Through this, it was found that BM03 produced lower levels of neutralizing antibody compared to Bsflagellin WT, and consequently exhibits higher TLR5-specific NF-kB activation activity.

### 2. Stability Evaluation

Certain proteins that showed excellent effects in the results of the immunogenicity evaluation were selected for stability evaluation, and the results are shown in Table 4 below.

TABLE 4

| | Stability after 2 weeks storage at 37° C. (Protein |
|---|---|
| Name | remaining % compared to initial of experiment) |
| Bsflagellin(WT) | 46.7 |
| BM03 | 82.2 |
| BM08 | 100 |
| BM11 | 100 |
| BM12 | 100 |
| BM13 | 99 |
| BM14 | 99.3 |

### 3. Productivity Evaluation

Certain proteins that showed excellent effects in the results of the immunogenicity evaluation were selected for productivity evaluation, and the results are shown in Table 5 below.

TABLE 5

| Name | Productivity (% compared to WT Bsflagellin) |
|---|---|
| BM03 | 250 |
| BM06 | 100 |
| BM08 | 336 |
| BM10 | 104 |
| BM11 | 273.87 |
| BM12 | 470 |
| BM13 | 408 |
| BM14 | 290 |
| BM15 | 420 |
| BM16 | 400 |
| BM18 | 120 |
| BS02 | 201.25 |

### INDUSTRIAL APPLICABILITY

The Bacillus subtilis flagellin variant provided by the present invention not only has reduced immunogenicity in the body compared to the wild-type, but also has improved storage stability, and thus it can be very useful in developing a treatment and/or vaccine adjuvant for diseases that can be prevented, improved, or treated by TLR5 activation, and highly industrially applicable.

What is claimed is:

1. A Bacillus subtilis flagellin variant comprising an amino acid sequence having at least 50% identity to a wild-type Bacillus subtilis flagellin of SEQ ID NO: 1 and having reduced immunogenicity compared to the wild-type Bacillus subtilis flagellin.

2. The Bacillus subtilis flagellin variant according to claim 1, characterized in that the residual rate of the protein is increased after storage for 2 weeks at 37° C. compared to the wild-type Bacillus subtilis flagellin.

3. The Bacillus subtilis flagellin variant according to claim 1, characterized by comprising one or more of the following mutations (1) to (7) in the amino acid sequence of the wild-type Bacillus subtilis flagellin of SEQ ID NO: 1:
(1) an acid substitution at one or more positions selected from the group consisting of L34, S49, M52, L59, A85, 186, Q103, Q104, L118, G151, G155, D176, G224, E231, D236, D238, M239 and R275;
(2) a deletion of the amino-terminal 0 domain (ND0, amino acids 131);
(3) a deletion of one or more amino acids within the amino-terminal 1 domain (ND1, amino acids 43-181);
(4) a deletion of one or more amino acids within the ND0-ND1 interface region (amino acids 32-42);
(5) a deletion of one or more amino acids within the carboxy-terminal 1 domain (CD1, amino acids 182-235);
(6) a deletion of one or more amino acids within the carboxy-terminal 0 domain (CD0, amino acids 239-275); and
(7) a deletion of the CD1-CD0 interface region (amino acids 236-238).

4. The Bacillus subtilis flagellin variant according to claim 3, characterized in that the amino acid substitution of (1) is one or more selected from the group consisting of of L34C, S49C, M52C, L59C, A85K, A85N, I86N, Q103A, Q104A, L118R, G151A, G155A, D176A, G224C, E231C, D236C, D238A, M239A and R275Q.

5. The Bacillus subtilis flagellin variant according to claim 4, characterized in that the deletion of one or more amino acids within ND1 of (3) is one or more deletions selected from the group consisting of a deletion of 43 a deletion of 43-50, a deletion of 100, a deletion of 181, a deletion of 144-146, a deletion of 164-179, and a deletion of 152-181 in the amino acid sequence of the wild-type Bacillus subtilis flagellin of SEQ ID NO: 1.

6. The Bacillus subtilis flagellin variant according to claim 3, characterized in that the deletion of one or more amino acids within the ND0-ND1 interface region of (4) is one deletion selected from the group consisting of a deletion of 32-33, a deletion of 32-41, and a deletion of 32-42 in the amino acid sequence of the wild-type Bacillus subtilis flagellin of SEQ ID NO: 1.

7. The Bacillus subtilis flagellin variant according to claim 3, characterized in that the deletion of one or more amino acids within CD1 of (5) is one or more deletions selected from the group consisting of a deletion of 182-183, a deletion of 182-186, and a deletion of 230-235.

8. The Bacillus subtilis flagellin variant according to claim 3, characterized in that the deletion of one or more amino acids within CD0 of (6) is one or more deletions selected from the group consisting of a deletion of 266-275, a deletion of 270-275, a deletion of 239-253, a deletion of 243-253, a deletion of 243-260, and a deletion of 239-275.

9. The Bacillus subtilis flagellin variant according to claim 3, characterized in that the variant comprises the mutations of (2), (3), and (5), and may or may not comprise the mutation of (1).

10. The Bacillus subtilis flagellin variant according to claim 3, characterized in that the variant comprises the mutations of (2), (3), and (5), and may or may not comprise the mutation of (1).

11. The Bacillus subtilis flagellin variant according to claim 1, characterized in that the Bacillus subtilis flagellin variant is selected from the following variants:
A85K, A85N, 186N, L118R, or R275Q in SEQ ID NO: 1;
a deletion of 1-31 in SEQ ID NO: 1;
deletions of T100, D144, G145, F146, A181, A182, and T183 in SEQ ID NO: 1;
deletions of 1-31, T100, D144, G145, F146, A181, A182, and T183 in SEQ ID NO: 1;
deletions of 1-31, T100, D144, G145, F146, D179, F180, A181, A182, and T183 in SEQ ID NO: 1;
deletions of 1-33, T100, D144, G145, F146, A181, A182, and T183 in SEQ ID NO: 1;
deletions of 1-41, T100, D144, G145, F146, A181, A182, and T183 in SEQ ID NO: 1;
deletions of 1-43, T100, D144, G145, F146, A181, A182, and T183 in SEQ ID NO: 1;
deletions of 1-50, T100, D144, G145, F146, A181, A182, and T183 in SEQ ID NO: 1;
deletions of 1-31, 164-179, T100, D144, G145, F146, A181, A182, and T183 in SEQ ID NO: 1;
deletions of 1-31, 152-186, T100, D144, G145, and F146 in SEQ ID NO: 1;
deletions of 1-31, 270-275, T100, D144, G145, F146, A181, A182, and T183 in SEQ ID NO: 1;
deletions of 1-31, 266-275, T100, D144, G145, F146, A181, A182, and T183 in SEQ ID NO: 1;
deletions of 1-31, 236-275, T100, D144, G145, F146, A181, A182, and T183 in SEQ ID NO: 1;
deletions of 1-31, 230-275, T100, D144, G145, F146, A181, A182, and T183 in SEQ ID NO: 1;
deletions of 1-50, 236-275, T100, D144, G145, F146, A181, A182, and T183 in SEQ ID NO: 1;
deletions of 1-31, 243-253, T100, D144, G145, F146, A181, A182, and T183 in SEQ ID NO: 1;
deletions of 1-31, 239-253, T100, D144, G145, F146, A181, A182, and T183 in SEQ ID NO: 1;
deletions of 1-31, 243-260, T100, D144, G145, F146, A181, A182, and T183 in SEQ ID NO: 1;
deletions of 1-31, 236-253, T100, D144, G145, F146, A181, A182 and T183 in SEQ ID NO: 1;
deletions of 1-31, T100, D144, G145, F146, A181, A182, and T183, and L34C and D236C in SEQ ID NO: 1;
deletions of 1-31, T100, D144, G145, F146, A181, A182, and T183, and S49C and E231C in SEQ ID NO: 1;
deletions of 1-31, T100, D144, G145, F146, A181, A182, and T183, and M52C and E231C in SEQ ID NO: 1;
deletions of 1-31, T100, D144, G145, F146, A181, A182, and T183, and L59C and G224C in SEQ ID NO: 1;
deletions of 1-31, T100, D144, G145, F146, A181, A182, and T183, and Q103A and Q104A in SEQ ID NO: 1;
deletions of 1-31, T100, D144, G145, F146, A181, A182, and T183, and G151A in SEQ ID NO: 1;
deletions of 1-31, T100, D144, G145, F146, A181, A182, and T183, and G155A in SEQ ID NO: 1;
deletions of 1-31, T100, D144, G145, F146, A181, A182, and T183, and D176A in SEQ ID NO: 1;
deletions of 1-31, T100, D144, G145, F146, A181, A182, and T183, and D238A in SEQ ID NO: 1;
deletions of 1-31, T100, D144, G145, F146, A181, A182, and T183, and Q103A, Q104A, and G155A in SEQ ID NO: 1;
deletions of 1-31, T100, D144, G145, F146, A181, A182, and T183, and Q103A, Q104A, and D176A in SEQ ID NO: 1;
deletions of 1-31, T100, D144, G145, F146, A181, A182, and T183, and Q103A, Q104A, and D238A in SEQ ID NO: 1;
deletions of 1-31, T100, D144, G145, F146, A181, A182, and T183, and Q103A, Q104A, G151A, and D176A in SEQ ID NO: 1;
deletions of 1-31, T100, D144, G145, F146, A181, A182, and T183, and Q103A, Q104A, G151A, and M239A in SEQ ID NO: 1;
deletions of 1-31, T100, D144, G145, F146, A181, A182, and T183, and A85N in SEQ ID NO: 1;
deletions of 1-31, T100, D144, G145, F146, A181, A182, and T183, and R275Q in SEQ ID NO: 1;
deletions of 1-31, T100, D144, G145, F146, A181, A182, and T183, and A85N and R275Q in SEQ ID NO: 1;
deletions of 1-31, T100, D144, G145, F146, D179, F180, A181, A182, and T183, and A85N in SEQ ID NO: 1;
deletions of 1-31, T100, D144, G145, F146, D179, F180, A181, A182, and T183, and R275Q in SEQ ID NO: 1; and
deletions of 1-31, T100, D144, G145, F146, D179, F180, A181, A182, and T183, and A85N and R275Q in SEQ ID NO: 1.

12. The Bacillus subtilis flagellin variant according to claim 1, characterized in that the flagellin variant retains NF-κB signaling activity.

13. A polynucleotide comprising a nucleotide sequence encoding the Bacillus subtilis flagellin variant according to claim 1.

14. A vector comprising the polynucleotide of claim 13.

15. A pharmaceutical composition for preventing or treating damage caused by radiation exposure; for preventing or treating reperfusion injury; for preventing or treating inflammatory bowel disease; for preventing or treating autoimmune disease; for preventing or treating viral infection; for preventing or treating metabolic disease; for preventing or treating aging; for enhancing immune function; for preventing or treating cancer; for preventing or treating liver disease; or for preventing or treating colitis, comprising the Bacillus subtilis flagellin variant according to claim 1 as an active ingredient.

16. A vaccine adjuvant comprising the Bacillus subtilis flagellin variant according to claim 1 as an active ingredient.

17. A use of the Bacillus subtilis flagellin variant according to claim 1 for manufacturing a pharmaceutical composition for treating damage caused by radiation exposure; for treating reperfusion injury; for treating inflammatory bowel disease; for treating autoimmune disease; for treating viral infection; for treating metabolic disease; for treating aging; for enhancing immune function; for treating cancer; for treating liver disease; or for treating colitis.

18. A method for treating damage caused by radiation exposure; treating reperfusion injury; treating inflammatory bowel disease; treating autoimmune disease; treating viral infection; treating metabolic disease; treating aging; enhancing immune function; treating cancer; treating liver disease; or treating colitis, comprising administering an effective amount of a pharmaceutical composition comprising the Bacillus subtilis flagellin variant according to claim 1 as an active ingredient to a subject in need thereof.
